SEQUENCE LISTING (44 sequences)

SEQ ID NO: 1 - protein, 317 aa, Campylobacter sp.
MRKDLVIIRVDGGIASQIAFVALGLEFESRGYKVKYDLSWFDEFGNGFYNEQNGYDRSYNITFDIPKAFPDIKCEIASRDEIEIYRKKYFVDNEEVIELKPPLYVGGYLGRHFDFKYRDFFAKHFNPKEIQESDSECKILLDNILSNHSCGVHIRMGDLSKEHVVYGKPLSVEYFLKNIQLVLQIEPKSKFYFFSDDLEWVKKKVIPSLHSIEYEICNVNSVEKGYLDLYLLSRCKIIIASQGSMGKYAKMLALDSKTLLITPNKNAPMENMMMVNWADSYESFEEKQIQKPLSWRNKIRLKIYKKLRNKLLQKGIL

SEQ ID NO: 2 - protein, 649 aa, Parabacteroides merdae
MKHETPVLSFIIPIYNVELYLPACLESVLHLPLTNFEIILIDDGSTDNSGRIADKYAENYSHIHVIHQTNAGLSATRNRGVELAEGNYIAFLDSDDWLIGDQIVDIYLQTSQANADMGMGNSLFCYPDGSNGNPFAPIPQNLIGQTIDGKYCFTELMKQNVYPPMVYNYIYRREWLLSHQLRFESVLHEDELWTPIALCYARRIVVTNIDFYGYRQREGSIMTSLRREKRIHDLFFIADRLIRLAENYRFDSDDKNLKSQLYVRSYLLYKIAFSSLAHIHDTRFILPTHHLYQLFAIKNKLTADARNTCINQYKTAKQGLKSYLKWRNSYWIRKLTYEISPETKLILIFNTMWNKPLDIPIQKIPANCIITTDRRFLSTADFVVFHLPDLSNELEEDLDKPEGQKWVAWSLECEENYHFLKDPEFMEVFDYKMSYHQDADIVYPYYRYEYRKELILMPEFSFHEKKDICMMISSPFNQSGRQEYLQELMQYISIDSYGKLFNNCKLEKDSGQDSKMELYRQYKFVIAFENSRANDYVTEKFYDPLLAGAVPIYLGAPNIETFAPGENCSVDIRNFSSPKELAAFLIEACKNEKIYNKFLQWKKEPLKPEFMKKIEAQRVHPFIRLCHLLHLPPPTSRYDGARKKNAHRS

SEQ ID NO: 3 - protein, 317 aa, Helicobacter pullorum
MRKDLVIIRVDGGIASQISFVALGLEFESRGYKVKYDLSWFDEFGNGICNEQNGYDRSYNIVFDIPKAFPDIKCEIASRDEIEIYRKKYFVDNEEVIELKPPLYVGGYLGRHFNFKYRDFFAKHFNPKEIQESDSECKILLDNILSNHSCGVHIRRGDLSKEHVVYGKPLSVEYFLKNIQLVLQIEPKSKFYFFGDDLEWVKKKVIPSLNSIEYEICNVNSVEKGYLDLYLLSRCKIIIASQGSMGKFAKMLASDTETLLIAPNKNAPMENMIMVNWVDSYESSGEKQIQKPLSWRNKIRLKIYKKFRNKLLQKGIL

SEQ ID NO: 4 - protein, 302 aa, Mediterranea sp.
MRKILILFYNKLWGVSWENEACASIPDGFELTDDRSRMAEADAVVFHLPDLARCMDGEEIEKRDGQLWVTWNLECDDNYPWVEAPEIRDLFDLRIGYRQQDDVWYPYFSFIRAEAFTRLPPRRPPLDKACLFVSSPFNRSHRFEFLRELMRHTEVDSFGRMLHNKDLPADEGRKTLMEKIADYKFVIGVENALARDYVTEKFFQPLLAGTVPVYRGAPNVEEFAPGRHCFVDARAFRSPADLAGFMNRCYEHPALYDELTAWRTQPLQPGFVQKLEQVKEHPFVRLCRVVEEKIRRIEYKIT

SEQ ID NO: 5 - protein, 464 aa, Helicobacter acinonychis
MFQPLLDAYTDSAYSDASDHKPPLNIAIANGWGGTKGFEASVLYFILSWRYKIALHENPNKPADLVFSNPLGQARKILSYQNTKRVFYTGENEVPNFNLFDYAIGFDGLDFKERYLRMPLYYASLHDKAQSVNDTTAPYTLKSDSLYALKKPAHCFKEDHPKLCSVVKGESDPFKRGFASFVAGNANAPMRNAFCEALNSVEPVTGGGAVKNTLGYNVTNKSEFLSQYKFNLCFENAQGYGYVTEKIIDAYFSHTIPIYWGSPSVAQDFNPKSFVNVHDFKDFDGAIDYIRYLHTHENAYLDMLYENPLNVIDGKACFYQDLSFKKILDFFKTILENDTIYHNNPFVFDRDLHEPLVSIDNLRADLLLLKDNYDGLKTDYDGLKTDYDGLKTDYDGLKTDYDGLKTDYDGLKTDYDGLKTDYDHLFKSALPLLELSQTTSFKIYHKIYQKTLPLLRMARKLVKK

SEQ ID NO: 6 - protein, 476 aa, Helicobacter pylori
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYDDLRVNYDDLRVNYDDLRVNYDDLRVNYDDLRVNYDDLRVNYDDLRVNYDRLLQNASPLLELSQNTTFKIYRKAYQKSLPLLRTIRRWVKK

SEQ ID NO: 7 - DNA, 954 bp, synthetic construct
  source 1..954: mol_type = other DNA
atgcgtaaagatctggtgattattcgtgtggatggtggtattgcaagccagattgcatttgttgcactgggcttagaatttgaaagccgtggttataaagtgaagtatgatctgagctggtttgatgaatttggcaacggcttttataacgagcagaatggttatgatcgcagctataacatcaccttcgatattccgaaagcattcccggatatcaaatgtgaaattgccagccgtgatgagattgaaatctatcgcaaaaagtacttcgtggacaacgaagaagtgatcgaactgaaaccgcctctgtatgttggtggttatctgggtcgtcatttcgatttcaaatatcgcgatttcttcgccaaacactttaacccgaaagaaatccaagaaagcgatagcgaatgtaaaatcctgctggataatatcctgagcaatcatagctgtggtgtgcatattcgtatgggtgatctgagtaaagaacatgtggtttatggtaaaccgctgagcgttgaatatttcctgaaaaacattcagctggtgctgcagattgaaccgaaaagcaaattctatttcttcagcgacgatctggaatgggtgaaaaagaaagttattccgagcctgcatagcatcgaatatgaaatttgcaatgtgaacagcgtggaaaaaggctatctggatctgtatctgctgagccgttgtaaaatcattattgcgagccagggtagcatgggtaaatatgcaaaaatgctggcactggatagcaaaaccctgctgattaccccgaataaaaacgcaccgatggaaaatatgatgatggtgaattgggcagatagctatgaaagctttgaagaaaagcagattcagaaaccgctgtcatggcgtaacaaaattcgtctgaaaatctacaaaaagctgcgcaataaactgctgcagaaaggcattctgtaa

SEQ ID NO: 8 - DNA, 1950 bp, synthetic construct
  source 1..1950: mol_type = other DNA
atgaaacatgaaacaccggtgctgagctttattatcccgatttataacgtggaactgtatctgcctgcatgtctggaaagcgttctgcatctgccgctgaccaattttgaaattattctgattgatgatggcagcaccgataatagcggtcgtattgcagataaatatgccgaaaactatagccacatccatgtgattcatcagaccaatgcaggtctgagcgcaacccgtaatcgtggtgttgaactggcagaaggtaactatattgcatttctggatagtgatgattggctgattggtgatcagatcgttgatatctatctgcagaccagccaggcaaatgcagatatgggtatgggtaatagcctgttttgttatccggatggtagcaatggtaatccgtttgcaccgattccgcagaatctgattggtcagaccattgatggtaaatattgctttaccgagctgatgaagcagaatgtttatccgcctatggtgtacaactatatctatcgtcgtgaatggctgctgagccatcagctgcgttttgaatcagttctgcatgaagatgaactgtggaccccgattgcactgtgttatgcacgtcgtattgttgtgaccaacatcgatttttatggttatcgtcagcgtgaaggtagcattatgaccagcctgcgtcgtgaaaaacgtattcatgacctgtttttcattgccgatcgtctgattcgtctggcagaaaattatcgctttgatagcgacgacaaaaacctgaaaagccagctgtatgttcgtagctatctgctgtataaaatcgcatttagcagcctggcacatattcatgatacccgttttattctgccgacgcatcatctgtatcagctgtttgccattaaaaacaaactgaccgcagatgcacgtaatacctgcattaatcagtataaaaccgcaaagcagggtctgaaaagctatctgaaatggcgtaacagctattggattcgcaaactgacgtatgaaattagtccggaaaccaaactgatcctgatctttaataccatgtggaacaaaccgctggatattccgattcagaaaattccggcaaactgcattattaccaccgatcgtcgttttctgagcaccgcagattttgttgtgtttcatctgccggatctgagcaatgaactggaagaggatctggataaaccggaaggtcagaaatgggttgcatggtcactggaatgtgaagaaaactatcactttctgaaagatccggaatttatggaagtgtttgactataagatgagctatcatcaggatgccgatatcgtgtatccgtattatcgttatgagtaccgcaaagaactgatactgatgcccgaatttagcttccacgaaaagaaagacatttgcatgatgattagcagcccgtttaatcagagcggtcgtcaagaatatctgcaagagctgatgcagtatatcagcattgatagctatggcaagctgttcaataactgcaaactggaaaaagatagcggtcaggatagcaagatggaattgtatcgtcagtacaaattcgtgatcgcctttgaaaattcacgtgccaatgattatgtgaccgagaaattctatgatccgctgctggcaggcgcagttccgatttatctgggtgcaccgaatattgaaacctttgcaccgggtgaaaattgcagcgttgatattcgtaatttcagcagcccgaaagagctggcagcatttctgatcgaagcatgcaaaaacgagaagatctacaacaaatttctgcagtggaagaaagaaccgctgaaacctgaattcatgaaaaagattgaagcccagcgtgtgcatccgtttattcgtctgtgtcatctgctgcatcttccgcctccgaccagccgttatgatggtgcacgtaaaaagaatgcacatcgtagctaa

SEQ ID NO: 9 - DNA, 954 bp, synthetic construct
  source 1..954: mol_type = other DNA
atgcgtaaagatctggtgattattcgtgtggatggtggtattgcaagccagattagctttgttgcactgggcttagaatttgaaagccgtggttataaagtgaagtatgatctgagctggtttgatgaatttggcaatggcatttgcaatgagcagaatggttatgatcgcagctataacatcgtgttcgatattccgaaagcattcccggatatcaaatgtgaaattgccagccgtgatgagattgaaatctatcgcaaaaagtacttcgtggacaacgaagaagtgatcgaactgaaaccgcctctgtatgttggtggttatctgggtcgtcatttcaacttcaaatatcgcgatttcttcgccaaacactttaacccgaaagaaatccaagaaagcgatagcgaatgtaaaatcctgctggataatatcctgagcaatcatagctgtggtgttcatattcgtcgtggtgatctgagtaaagaacatgtggtttatggtaaaccgctgagcgttgaatatttcctgaaaaacattcagctggtgctgcagattgaaccgaaaagcaaattctatttctttggcgacgatctggaatgggtgaaaaagaaagttattccgagcctgaacagcatcgaatatgagatttgtaatgtgaacagcgtggaaaaaggctatctggatctgtatctgctgagccgttgtaaaatcattattgcgagccagggtagcatgggtaaatttgcaaaaatgctggcaagcgataccgaaacactgctgattgcaccgaataaaaacgcaccgatggaaaacatgattatggtgaattgggtcgatagctatgaaagcagcggtgaaaaacaaattcagaaaccgctgtcatggcgtaacaaaattcgcctgaaaatctacaaaaagtttcgcaacaaactgctgcagaaaggcattctgtaa

SEQ ID NO: 10 - DNA, 909 bp, synthetic construct
  source 1..909: mol_type = other DNA
atgcgcaaaatcctgatcctgttctataacaaactgtggggtgttagctgggaaaatgaagcatgtgcaagcattccggatggttttgaactgaccgatgatcgtagccgtatggccgaagcagatgcagttgtttttcatctgccggatctggcacgttgtatggatggtgaagaaattgaaaaacgtgatggtcagctgtgggttacctggaatctggaatgtgatgataactatccgtgggttgaagcaccggaaattcgtgacctgtttgatctgcgtattggttatcgtcagcaggatgatgtttggtatccgtattttagctttattcgtgccgaagcgtttacccgtctgcctccgcgtcgtccgcctctggataaagcatgtctgtttgttagcagcccgtttaatcgtagccatcgttttgaatttctgcgtgaactgatgcgtcataccgaagttgatagctttggtcgtatgctgcataataaagatctgcctgcagatgaaggtcgtaaaaccctgatggaaaagattgccgattataagtttgtgatcggtgttgaaaatgcactggcacgtgattatgttaccgaaaaattctttcagccgctgctggcaggcaccgttccggtttatcgtggtgcaccgaatgttgaagaatttgcaccgggtcgtcattgttttgttgatgcacgtgcctttcgttcaccggcagatctggcaggctttatgaatcgttgttatgaacatccggcactgtatgatgaactgacagcatggcgtacccagcctctgcagcctggttttgttcagaaactggaacaggttaaagaacatccgtttgttcgtctgtgtcgtgttgttgaagagaaaattcgtcgcatcgagtacaaaatcacctaa

SEQ ID NO: 11 - DNA, 1395 bp, synthetic construct
  source 1..1395: mol_type = other DNA
atgtttcagccgctgctggatgcatataccgatagcgcatatagtgatgcaagcgatcataaaccgcctctgaatattgcaattgccaatggttggggtggcaccaaaggttttgaagcaagcgttctgtattttatcctgagctggcgctataaaatcgccctgcatgaaaatccgaataaaccggcagatctggtttttagcaatccgctgggtcaagcacgtaaaattctgagctatcagaataccaaacgcgtgttttataccggtgaaaatgaagtgccgaacttcaacctgtttgattatgccattggttttgatggcctggatttcaaagaacgttatctgcgtatgccgctgtattatgcaagcctgcatgataaagcacagagcgttaatgataccaccgcaccgtatacactgaaaagcgatagcctgtatgcactgaagaaaccggcacattgttttaaagaggatcatccgaaactgtgcagcgttgttaaaggtgaaagcgatccttttaaacgtggttttgcaagctttgttgcaggtaatgcaaatgcaccgatgcgtaatgcattttgtgaagcactgaatagcgttgaaccggttaccggtggtggtgcagtgaaaaacaccctgggttataatgttaccaacaaaagcgaattcctgagccagtataaattcaatctgtgcttcgaaaatgcccaaggttatggttatgtgaccgagaaaattatcgatgcctatttcagccataccattccgatttattggggtagcccgagcgttgcacaggatttcaatccgaaaagctttgtgaacgtgcacgacttcaaagattttgatggtgccattgattatatccgctatctgcatacccatgaaaatgcctatctggatatgctgtatgaaaacccgctgaatgtgattgatggtaaagcctgtttttatcaggatctgagcttcaaaaagatcctggactttttcaaaaccatcctggaaaacgataccatctatcataacaacccgtttgtgtttgatcgtgatctgcatgaaccgctggttagcattgataatctgcgtgccgatctgctgctgctgaaagataattatgatggtctgaaaaccgattatgatggccttaagacagattatgatgggttaaaaacagactacgatggcttgaaaacggattatgatggactgaaaacggactacgacgggttaaagaccgattatgatggtttaaagaccgattacgaccacctgtttaaaagcgcactgcctctgctggaactgagccagaccacaagctttaaaatctaccacaagatctaccagaaaaccctgccactgctgcgtatggcacgtaaactggttaaaaagtaa

SEQ ID NO: 12 - DNA, 6706 bp, synthetic construct
  source 1..6706: mol_type = other DNA
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaatgatgtttttacgtcaggaagactttgccacggtagtgcgctccactccgcttgtctctctcgactttattgtcgagaacagtcgcggcgagtttctgcttggcaaaagaaccaaccgcccggcgcagggttactggtttgtgccgggagggcgcgtgcagaaagacgaaacgctggaagccgcatttgagcggctgacgatggcggaactggggctgcgtttgccgataacagcaggccagttttacggtgtctggcagcacttttatgacgataacttctctggcacggatttcaccactcactatgtggtgctcggttttcgcttcagagtatcggaagaagagctgttactgccggatgagcagcatgacgattaccgctggctgacgtcggacgcgctgctcgccagtgataatgttcatgctaacagccgcgcctattttctcgctgagaagcgtaccggagtacccggattatgaaaatactggtctacggcattaactactcgccggagttaaccggcatcggcaaatacaccggcgagatggtggaatggctggcggcacaaggtcatgaggtgcgggtcattaccgcaccgccttactacccgcaatggcaggtgggcgagaactattccgcctggcgctacaaacgagaagagggggccgccacggtgtggcgctgcccgctgtatgtgccaaaacagccgagcaccctgaaacgcctgttgcatctgggcagttttgccgtcagcagtttctttccgctgatggcgcaacgtcgctggaagccggatcgcattattggcgtggtgccaacgctgttttgcgcgccgggaatgcgcctgctggcgaaactctctggtgcgcgtaccgtgctgcatattcaggattacgaagtggacgccatgctggggctgggccttgccggaaaaggcaaaggcggcaaagtggcacagctggcaacggcgttcgaacgtagcggactgcataacgtcgataacgtctccacgatttcgcgttcgatgatgaataaagccatcgaaaaaggcgtggcggcggaaaacgtcatcttcttccccaactggtcggaaattgcccgttttcagcatgttgcagatgccgatgttgatgcccttcgtaaccagcttgacctgccggataacaaaaaaatcattctttactccggcaatattggtgaaaagcaggggctggaaaacgttattgaagctgccgatcgtctgcgcgatgaaccgctgatttttgccattgtcgggcagggcggcggcaaagcgcggctggaaaaaatggcgcagcagcgtggactgcgcaacatgcaatttttcccgctgcaatcgtatgacgctttacccgcactgctgaagatgggcgattgccatctggtggtgcaaaaacgcggcgcggcagatgccgtattgccgtcgaaactgaccaatattctggcagtaggcggtaacgcggtgattactgctgaagcctacacagaactggggcagctttgcgaaacctttccgggcattgcggtttgcgttgaaccggaatcggtcgaggcgctggtggcggggatccgtcaggcgctcctgctgcccaaacacaacacggtggcacgtgaatatgccgaacgcacgctcgataaagagaacgtgttacgtcaatttataaatgatattcggggataattatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 13 - protein, 332 aa, Neisseria meningitidis
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 14 - protein, 273 aa, Helicobacter pylori
MRVFAISLNQKVCDTFGLVFRDTTTLLNSINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYIRLMHLLYDASVKSEPLSHKNHEIQERVGIIKAYSEGVGTQGYVITPKIAKVFLKCSRKWVVPVDTIMDATFIHGVKNLVLQPFVIADDEQISTIARKEEPYSPKIALMRELHFKYLKYWQFV

SEQ ID NO: 15 - protein, 417 aa, Escherichia coli [sequence omitted]

SEQ ID NO: 16 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 17 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 18 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 19 - DNA, 141 bp, synthetic construct
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 20 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 21 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 22 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 23 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 24 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 25 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 26 - DNA, 189 bp, synthetic construct
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 27 - DNA, 239 bp, synthetic construct
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 28 - DNA, 300 bp, Escherichia coli
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 29 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 30 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 31 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 32 - DNA, 350 bp, synthetic construct
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 33 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 34 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 35 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 36 - DNA, 189 bp, synthetic construct; identical to SEQ ID NO: 26
  source 1..189: mol_type = other DNA

SEQ ID NO: 37 - DNA, 107 bp, Escherichia coli
  source 1..107: mol_type = other DNA
tgtgagttagctcactcattaggcaccccaggctttacactttatgcttccggctcgtatgttgtgtggaattgtgagcggataacaatttcacacaggaaacagct

SEQ ID NO: 38 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 39 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 40 - DNA, 1431 bp, synthetic construct
  source 1..1431: mol_type = other DNA
atgtttcagccgctgctggatgcatttattgaaagcgcaagcattgagaaaatggtgagcaaaagccctccgcctccgctgaaaattgcagttgcaaattggtggggtgatgaagaaatcaaagagtttaaaaagagcgtcctgtactttattctgagccagcgttatgcaattaccctgcatcagaatccgaatgaaagcagcgatctggtttttagcaatccgctgggtgcagcacgtaaaattctgagctatcagaataccaaacgcgtgttttataccggtgaaaatgaaagcccgaacttcaacctgtttgattatgccattggtttcgatgagctggattttaatgatcgttatctgcgtatgccgctgtattatgcacatctgcattatgaagcagaactggttaatgataccaccgcaccgtataaactgaaagataatagcctgtatgcactgaaaaagccgagccatcattttaaagaaaatcatccgaatctgtgtgccgtggtgaatgatgaaagcgatctgctgaaacgtggttttgcaagctttgttgcaagcaatgcaaatgcaccgatgcgtaatgcattttatgatgccctgaatagcattgaaccggttaccggtggtggtagcgttcgtaataccctgggttataaagttggcaacaaaagcgaattcctgagccagtataaattcaatctgtgctttgaaaacagccaaggctatggttatgtgaccgagaaaattctggatgcctatttcagccataccattccgatttattggggtagcccgagcgttgcaaaagatttcaatccgaaaagctttgtgaacgtgcacgatttcaacaactttgatgaagccatcgactacatcaaatatctgcatacccatccgaatgcctatctggatatgctgtatgaaaatccgctgaatacactggatggtaaagcctatttctatcaggatctgagcttcaaaaagatcctggactttttcaaaaccatcctggaaaacgataccatctatcacaataacccgttcatcttttatcgcgatctgcacgaaccgctgattagtattgatgatctgcgtgtgaattatgatgacctgcgcgttaactacgatgatttacgcgttaattatgatgatcttcgggtcaactatgatgacttaagagtgaactacgacgatctgagagtcaattatgatgatttgagagttaattatgatgacttgcgtgtgaattatgatgatttgcgggtgaactacgacgacctgcgtgttaactatgatcgtctgctgcagaatgcaagccctctgctggaactgagccagaatacaacctttaaaatctatcgcaaggcctaccagaaaagcctgcctctgctgcgtaccattcgtcgttgggttaaaaagtaa

SEQ ID NO: 41 - protein, 320 aa, Helicobacter hepaticus
MKDDLVILHPDGGIASQIAFVALGLAFEQKGAKVKYDLSWFAEGAKGFWNPSNGYDKVYDITWDISKAFPALHIEIANEEEIERYKSKYLIDNDRVIDYAPPLYCYGYKGRIFHYLYAPFFAQSFAPKEAQDSHTPFAALLQEIESSPSPCGVHIRRGDLSQPHIVYGNPTSNEYFAKSIELMCLLHPQSSFYLFSDDLAFVKEQIVPLLKGKTYRICDVNNPSQGYLDLYLLSRCRNIIGSQGSMGEFAKVLSPHNPLLITPRYRNIFKEVENVMCVNWGESVQHPPLVCSAPPPLVSQLKRNAPLNSRLYKEKDNASA

SEQ ID NO: 42 - protein, 331 aa, Bacteroides fragilis
MCDCLSIILLVKMKKIYLKFVDFWDGFDTISNFIVDALSIQYEVVLSNEPDYLFYSCFGTSHLEYDCIKIMFIGENIVPDFNVCDYAIGFNYIDFGDRYLRLPLYAIYDGFSNLQNKKIDVNKALDRKFCSIVVSNNKWADPIRETFFKLLSSYKKVDSGGRAWNNIGGPVDNKLDFISQYKFNIAFENSRVLGYTTEKIMEPMQVNSIPVYWGNPLVGKDFNVDSFVNAHDFDSLERLVEYIIELDSSKDKYLEMLEKPWLLDKTYLDWKQLLLNFINNIMMKSYKDAKYLVNYGHAGKYRNEQRFWGRCERKFKLQRIIEYYSQLFDRK

SEQ ID NO: 43 - DNA, 996 bp, synthetic construct
  source 1..996: mol_type = other DNA
atgtgtgattgcctgagcattattctgctggtgaaaatgaaaaagatctatctgaagttcgtggatttctgggatggctttgataccattagcaactttattgttgatgccctgagcatccagtatgaagttgttctgagcaatgaaccggattacctgttttatagctgttttggcaccagccatctggaatatgattgcatcaaaatcatgttcatcggcgaaaatatcgtgccggattttaatgtttgcgattatgccatcggcttcaactatattgattttggtgatcgttatctgcgtctgccgctgtatgcaatttatgatggttttagcaatctgcagaacaaaaagatcgatgtgaataaagccctggatcgcaaattttgcagcattgttgtttccaacaacaaatgggcagatccgattcgtgaaacctttttcaaactgctgagcagctataagaaagttgatagcggtggtcgtgcatggaataacattggtggtccggttgataataaactggatttcatcagccagtacaaatttaacattgcctttgaaaatagccgtgtgctgggttataccaccgagaaaatcatggaaccgatgcaggttaatagcattccggtttattggggtaatccgctggttggcaaagatttcaatgttgatagctttgtgaacgcccacgattttgatagcctggaacgtctggttgaatatatcattgaactggatagcagcaaggacaagtatctggaaatgctggaaaaaccgtggctgctggataaaacatatctggattggaaacagctgctgctgaactttattaacaacattatgatgaaaagctataaagatgccaaatatctggtgaactatggccatgcaggtaaatatcgtaatgaacagcgtttttggggtcgttgtgaacgtaaattcaagctgcagcgtatcatcgagtattacagccagctgtttgatcgcaagtaa

SEQ ID NO: 44 - DNA, 963 bp, synthetic construct
  source 1..963: mol_type = other DNA
atgaaagatgatctggttatcctgcatcctgatggtggtattgcaagccagattgcatttgttgcactgggtttagcatttgaacagaaaggtgccaaagtgaaatatgacctgagctggtttgcagaaggtgcaaaaggtttttggaatccgagcaatggttatgacaaagtgtatgatattacctgggatatcagcaaagcatttccggcactgcatattgaaattgccaacgaagaagaaatcgagcgctacaaaagcaaatatctgattgataacgatcgcgtgattgattatgcaccgcctctgtattgttatggttataaaggtcgcatcttccactatctgtatgcaccgtttttcgcacagagctttgcaccgaaagaagcacaggatagtcataccccgtttgcagccctgctgcaagaaattgaaagcagcccgagtccgtgtggtgttcatattcgtcgtggtgatctgagccagccgcatattgtttatggtaatccgaccagcaatgagtattttgccaaaagcattgaactgatgtgtctgctgcatccgcagagcagcttttacctgtttagcgacgatctggcatttgtgaaagagcagattgttccgctgctgaaaggtaaaacctatcgtatttgtgatgtgaataatccgagtcagggttatctggatctgtatctgctgagccgttgtcgtaacattattggtagccagggtagcatgggtgaatttgcaaaagttctgagtccgcataatcctctgctgattacaccgcgttatcgcaacattttcaaagaagtggaaaatgtgatgtgcgtgaattggggtgaaagcgttcagcatccgcctctggtgtgtagcgcaccgccaccgctggttagccagctgaaacgtaatgcaccgctgaatagccgtctgtataaagaaaaagataatgccagcgcctaa

(19) United States
(12) Patent Application Publication
(10) Pub. No.: US 2026/0265800 A1
(43) Pub. Date: Sep. 10, 2026

(54) NEW FUCOSYLTRANSFERASES FOR IN VIVO SYNTHESIS OF LNFP-III

(71) Applicant: DSM IP Assets B.V., Maastricht (NL)

(72) Inventor: Manos PAPADAKIS, Hørsholm (DK)

(21) Appl. No.: 18/994,191

(22) PCT Filed: Jul. 14, 2023
(86) PCT No.: PCT/EP2023/069730

§ 371 Date: Jan. 14, 2025

(30) Foreign Application Priority Data
Jul. 15, 2022 (DK) PA202200689

(51) Int. Cl. C12P 19/04; C12N 9/10

(52) U.S. Cl. CPC C12P 19/04; C12N 9/1051; C12Y 204/01065

(57) ABSTRACT

The present disclosure relates to the production of fucosylated Human Milk Oligosaccharides (HMOs). In particular, it relates to alpha-1,3-fucosyltransferases for the production of lacto-N-fucopentaose III (LNFP-III), as well as to genetically engineered cells and their use in said production. Preferably the alpha-1,3-fucosyltransferases described herein do not fucosylate the reducing end of the acceptor oligosaccharide used as substrate for the alpha-1,3-fucosyltransferases.

### CROSS-REFERENCE TO RELATED APPLICATIONS

This application is a national stage entry pursuant to 35 U.S.C. § 371 of International Application No. PCT/EP2023/069730, filed on Jul. 14, 2023, which claims priority to Denmark Application No. PA202200689, filed on Jul. 15, 2022, the entire contents of all of which are hereby incorporated by reference in their entirety.

### SEQUENCE LISTING

The computer-readable Sequence Listing submitted on Apr. 29, 2026, and identified as follows: 65,330 bytes ST.26 XML document file named “Sequence_Listing.xml,” created Jan. 14, 2026, is incorporated herein by reference in its entirety.

### FIELD

The present disclosure relates to the production of fucosylated Human Milk Oligosaccharides (HMOs). In particular, it relates to alpha-1,3-fucosyltransferases for the production of lacto-N-fucopentaose III (LNFP-III), as well as to genetically engineered cells and their use in said production. Preferably the alpha-1,3-fucosyltransferases described herein do not fucosylate the reducing end of the acceptor oligosaccharide used as substrate for the alpha-1,3-fucosyltransferases.

### BACKGROUND

The design and construction of bacterial cell factories to produce fucosylated Human Milk Oligosaccharides (HMOs), especially for more complex fucosylated HMOs is of paramount importance to provide innovative and scalable solutions for the more complex products of tomorrow.

Production of complex fucosylated HMOs has e.g., been described in WO2016/040531, suggesting that the α-1,3-fucosyltransferase CafD can produce LNFP-III, and in WO2019/008133, describing the α-1,3-fucosyltransferase FucT 109 which appears to fucosylate both the glucose (Glc) and N-acetylglucosamine (GlcNAc) moiety of Lacto-N-neotetraose (LNnT), thus potentially generating a mixture containing all three of LNnT, LNFP-III and LNFP-VI.

Dumon et al., 2004 (Biotechnol. Prog. 2004, 20, 412-419) further describes an α-1,3-fucosyltransferase, FutB, which is also suggested to produce a mixture of LNnT, LNFP-III, LNFP-VI and LNDFH-III.

The fucosyltransferases disclosed in the prior art (CafD, FucT109 and FutB), however, only produce minor amounts, if any, of the complex fucosylated HMOs, with high by-product formation.

In summary, production of fucosylated HMOs, especially more complex fucosylated HMOs, is challenging due to the lack of fucosyltransferases with the desired substrate specificity, as well as low production yield of the desired fucosylated HMOs as compared to other HMO products present after fermentation, such as HMO precursor products, which may require laborious separation procedures.

### SUMMARY OF THE INVENTION

The need for highly substrate specific α-1,3-fucosyltransferases is solved by the identification of a selection of α-1,3-fucosyltransferases which exhibit low or no specificity for the glucose moiety in LNnT as a substrate for fucosylation reactions, but which are highly substrate specific for the N-acetylglucosamine (GlcNAc) moiety in LNnT, thus producing almost exclusively the complex fucosylated HMO LNFP-III, or mixtures of HMOs that comprise a high content of LNFP-III. The α-1,3-fucosyltransferases presented herein may therefore be used in the production of pure, or almost pure LNFP-III. Hence, provided herein are enzymes, mixtures, compositions, uses, genetically engineered cells and methods for the production of LNFP-III.

In a first aspect, the invention relates to a genetically engineered cell, which is capable of producing one or more fucosylated HMOs, preferably LNFP-III, and which comprises a recombinant nucleic acid sequence encoding a glycosyltransferase with α-1,3-fucosyltransferase activity, wherein the glycosyltransferase is selected from the group consisting of Camp1, Parm1 and Hpull1 with an amino acid sequence according to SEQ ID NO: 1, 2 and 3, or a functional homologue thereof with an amino acid sequence that is at least 80% identical to SEQ ID NO: 1, 2 and 3 [Camp1, Parm1 and Hpull1 amino acid].

In a second aspect, the invention relates to a method for producing one or more fucosylated HMOs, preferably LNFP-III, said method comprising culturing a genetically engineered cell capable of producing one or more fucosylated HMOs, comprising a recombinant nucleic acid sequence encoding an glycosyltransferase with α-1,3-fucosyltransferase activity, wherein the glycosyltransferase is selected from the group consisting of Camp1, Parm1, Hpull1 and Hacin2 with an amino acid sequence according to SEQ ID NO: 1, 2, 3, or 5, or a functional homologue thereof with an amino acid sequence that is at least 80% identical to SEQ ID NO: 1, 2, 3 or 5 [Camp1, Parm1, Hpull1 and Hacin2 amino acid].

In a third aspect, the invention relates to the use of an enzyme with α-1,3-fucosyltransferase activity, for the production of a fucosylated product, such as an HMO and preferably LNFP-III, wherein the enzyme is selected from the group consisting of Camp1, Parm1, Hpull1 and Hacin2 with an amino acid sequence according to SEQ ID NO: 1, 2, 3, or 5 or a functional homologue thereof which amino acid sequence is at least 80% identical to SEQ ID NO: 1, 2, 3 or 5 [Camp1, Parm1, Hpull1 and Hacin2 amino acid].

In a fourth aspect, the invention relates to a mixture of HMOs, consisting essentially of LNFP-III and LNnT, and with low amounts of 3-FL, LNDFH-III and/or pLNnH, such as below 10 molar % of the total molar HMO content in the mixture.

In some embodiments, the mixture of HMOs consists essentially of 30-99 molar % LNFP-III, 0-65 molar % LNnT, 0-5 molar % 3-FL and 0-10% pLNnH, in total adding up to 100% of the molar content of HMOs in the mixture.

A fifth aspect of the invention provides
a) a mixture of HMOs consisting essentially of LNFP-III and LNnT, and with low amounts of 3-FL or pLNnH, such as below 10% total molar HMO content in the composition, or
b) a mixture of HMOs consisting essentially of 30-99 molar % LNFP-III, 0-65 molar % LNnT, 0-20 molar % 3-FL, 0-5 molar % LNDFH-III and 0-10 molar % pLNnH, in total adding up to 100% molar content.

The invention further relates to compositions comprising the mixtures of a) and b), and use of said compositions in an infant formula, a dietary supplement, or medical nutrition.

### BRIEF DESCRIPTION OF THE FIGURES

FIG. 1 illustrates the data from the single copy strains of table 5, clearly showing the difference in LNFP-III production from the strains with the five novel enzymes compared to the prior art enzymes FutB, CafD and FucT109.

FIG. 2 is an illustration of the pathway for making LNFP-III and the potential by-products that can be generated in the process. The accepter for the alpha-1,3-fucosyltransferases can be lactose, LNnT, LNFP-VI and LNFP-III, depending on the specificity of the alpha-1,3-fucosyltransferase. Fucosylated by-products are for example 3FL, LNFP-VI, and LNDFH-III. Neutral by-products are for example LNT-II and LNnT. Depending on the genetically modified cell the initial substrate for the LNFP-III production may be lactose, LNT-II or even LNnT, as long as the strain can take up the initial substrate. In the figure β1,3-GlcNACT is a β1,3-glucosaminyltransferase, β1,3-GalT is a β1,3-galactosyltransferase, Glc α1,3-FucT is a α1,3-fucosyltransferase with activity on glucose moieties in the acceptor oligosaccharide, GlcNAc α1,3-FucT is a α1,3-fucosyltransferase with activity on the GlcNAc moiety in the acceptor oligosaccharide; GDP is guanosine-diphosphate and UDP is uridine-diphosphate.

FIG. 3: Shows the experimental setup of the regeneration and viability assessment of lyophilized probiotics under pH 3.0 acidic conditions.

FIG. 4: Shows the regeneration and viability of lyophilized Lactobacillus casei (DSM 32382), incubated for 3 h at pH 3.0 and plated in two dilutions 1:100 (E-2), 1:1000 (E-3) A) is the control without HMOs B) is Lactobacillus casei (DSM 32382) in combination with an HMO mixture containing 50% LNFP-III and 50% LNnT (mix1); C) is Lactobacillus casei (DSM 32382) in combination with an HMO mixture containing 75% LNFP-III and 25% LNnT (mix2); B) is Lactobacillus casei (DSM 32382) in combination with an HMO mixture containing 40% LNFP-III and 50% LNnT and 5% pLNnH (mix3).

FIG. 5: shows the results from the plates in FIG. 4 expressed as mean values (n=2) with standard deviation (SD) of colony-forming units (CFU) per milliliter calculated from Lactobacillus casei colonies on agar plates when plated at dilution step E-3, mix 1 and 2 showed statistically significant difference relative to control of p<0.01 and mix 3 showed statistically significant difference relative to control of p<0.05.

### DETAILED DESCRIPTION

The present invention approaches the biotechnological challenges of in vivo HMO production of, in particular, complex fucosylated HMOs which comprise at least five monosaccharides of which at least one saccharide moiety is a fucosyl moiety, such as the complex fucosylated HMO LNFP-III, which contains a single fucosyl moiety. The present disclosure offers specific strain engineering solutions to produce specific complex fucosylated HMOs, in particular LNFP-III, by exploiting the substrate specificity and activity of the 1,3-fucosyltransferases of the present disclosure towards the GlcNAc moiety in LNnT.

A genetically engineered cell of the present disclosure expresses genes encoding key enzymes for the biosynthesis of fucosylated HMOs. In addition, the genetically engineered cell may express additional genes needed to produce LNnT, either from lactose or LNT-II as the initial substrate. Alternatively, the cell may be engineered to take up LNnT (see for example WO2023/099680) only needing the 1,3-fucosyltransferase activity in the cell. In some embodiments, a genetically engineered cell of the present description further expresses one or more of the de novo GDP-fucose pathway genes, manA, manB, manC, gmd and/or wcaG, responsible for the formation of GDP-fucose. It may be advantageous to overexpress one or more of these genes and/or to upregulate the colanic acid gene cluster (CA), including the genes gmd, wcaG, wcaH, wcal, manC and manB from E. Coli, through introduction of a nucleic acid construct encoding the CA as shown in SEQ ID NO: 12, allowing for formation of GDP-fucose, which enables the cell to produce a higher level of fucosylated oligosaccharide from one or more oligosaccharide substrates, such as lactose, LNT-II and/or LNnT. Depending on the intended use of substrate, one or more additional glycosyltransferases and pathways for producing nucleotide-activated sugars, such as glucose-UDP-GlcNac, CMP-N-acetylneuraminic acid, UDP-galactose, UDP-glucose, UDP-N-acetylglucosamine, UDP-N-acetylgalactosamine and CMP-N-acetylneuraminic acid can also be present in the genetically engineered cell.

### Production of LNFP-III

The advantage of using any one of the α-1,3-fucosyltransferases of the present disclosure in the present context is their ability to specifically recognize and fucosylate the GlcNAc moiety in LNnT, to generate LNFP-III. In particular, the present disclosure describes enzymes with α-1,3-fucosyltransferase activity (α-1,3-fucosyltransferases) that are more active on the GlcNAc moiety of LNnT than α-1,3-fucosyltransferases described in the prior art, such as FucT109 (see WO2019/008133) and FutB (see Dumon et al., 2004). Furthermore, the α-1,3-fucosyltransferases described herein have very low activity on the glucose moieties of lactose and LNnT. If LNnT is available in sufficient amounts inside the genetically engineered cell, very little, if any, 3-FL is produced by the α-1,3-fucosyltransferases described in the present disclosure. The traits of the α-1,3-fucosyltransferases described herein are therefore well-suited for high-level industrial production of LNFP-III without production of high levels of alternatively fucosylated HMOs (side products), such as 3FL, LNFP-VI, LNDFH-III and other by-product HMOs or by-product non-HMO oligosaccharides.

In embodiments the α-1,3-fucosyltransferases of the present disclosure primarily fucosylates the N-acetylglucoseamine (GlcNAc) moiety of an acceptor oligosaccharide such as LNnT. The term primarily is to be understood as less than 5%, such as less than 4%, such as less than 3%, such as less than 2% of other moieties in LNnT are fucosylated by the α-1,3-fucosyltransferases of the present disclosure.

In embodiments the α-1,3-fucosyltransferases of the present disclosure has low or no activity on the glucose moiety in the reducing end of the acceptor molecule. The acceptor molecule is a saccharide or oligosaccharide such as for example lactose or an HMO such as LNT-II or LNnT, but also other HMOs. LNnT is the preferred acceptor molecule for the α-1,3-fucosyltransferases of the present disclosure. The term low or no activity is to be understood as less than 5%, such as less than 4%, such as less than 3%, such as less than 2% of the glucose moiety in the reducing end of the acceptor molecule is fucosylated by the α-1,3-fucosyltransferases of the present disclosure.

The genetically engineered cells of the present invention which express an α-1,3-fucosyltransferase with high specificity for the GlcNAc moiety in LNnT, enable the production of high titters of LNFP-III. In particular, in absence of other complex fucosylated HMOs, such as LNFP-VI and/or LNDFH-III. Thereby, the present invention enables a more efficient LNFP-III production, which is highly beneficial in biotechnological production of more complex fucosylated HMOs, such as LNFP-III, for example due to reduced purification needs.

Consequently, the mixtures of HMOs produced by the cells and/or methods described herein contain a high percentage of LNFP-III out of the total amount of HMOs produced, such as at least 30% of the total amount of HMOs, preferably at least 50%, such as at least 75%, such as alt least 80%, such as at least 90% of the total amount of HMO produced by the cell and/or method.

In the following sections, individual elements of the invention, and in particular of the genetically engineered cell are described. It is understood that these elements can be combined across the individual sections.

### Oligosaccharides

In the present context, the term “oligosaccharide” means a sugar polymer containing at least three monosaccharide units, i.e., a tri-, tetra-, penta-, hexa- or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. N-acetylneuraminic acid). Preferably, the oligosaccharide is an HMO.

### Human Milk Oligosaccharide (HMO)

Preferred oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an α-L-fucopyranosyl and/or an α-N-acetyl-neuraminyl (fucosyl) moiety. HMO structures are e.g., disclosed by Xi Chen in Chapter 4 of Advances in Carbohydrate Chemistry and Biochemistry 2015 vol 72.

The present invention focuses on fucosylated HMO's. Examples of fucosylated HMOs include, 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-fucopentaose VI (LNFP-VI), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II), fucosyl-lacto-N-neohexaose (FLNnH), 3-fucosyl-3′-fucosyllactose (FSL), fucosyl-LST-a (FLST-a), fucosyl-LST b (FLST b), fucosyl-LST-c (FLST-c), fucosyl-LST d (FLST-d) and fucosyl-lacto-N-hexaose (SLNH).

In the context of the present invention, complex fucosylated HMOs are fucosylated HMOs that comprises at least 5 monosaccharide units of which at least one monosaccharide unit is a fucosyl unit, non-limiting examples of complex fucosylated HMOs are the fucosylated HMOs consisting of 5 monosaccharide units e.g., LNFP-I, LNFP-II, LNFP-III, LNFP-V and LNFP-VI and fucosylated HMO with 6 monosaccharide units, such as but not limited to LNDFH-I, LNDFH-II and LNDFH-III. Preferably, a complex fucosylated HMO is one that require at least three different glycosyltransferase activities to be produced from lactose as the initial substrate, e.g., the formation of LNFP-III requires an α-1,3-fucosyltransferase, a β-1,3-N-acetyl-glucosaminyl-transferase and a β-1,4-galactosyltransferase.

In embodiments of the present invention, the fucosylated human milk oligosaccharide (HMO) produced by the cell is LNFP-III, such as exclusively or essentially exclusively LNFP-III. In a further embodiment of the present invention, at least 30%, such as at least 50%, 60%, 70%, 75%, 80%, 85%, 90%, 95%, 98% or 99% of the molar content of the total HMOs produced by said cell is LNFP-III. Preferably, at least 75% of the molar content of the total HMOs produced by said cell is LNFP-III.

In embodiments the genetically engineered cell described herein produces at least 50% LNFP-Ill of the molar content of the total HMOs produced by said cell and the remaining 50% of the total HMO produced by said cell is by-product HMOs selected from the group consisting of LNDFH-III, 3FL, LNnT and pLNnH.

In further embodiments, at least 90% of the molar content of the total HMOs produced by said cell is LNFP-III and LNnT. In additional embodiments of the invention, less than 1%, such as 0.0%, 0.1%, 0.2%, 0.3%, 0.4%, 0.5%, 0.6%, 0.7%, 0.8%, 0.9% or 0.99% of the total molar content of HMOs produced by the cell is LNFP-VI.

In additional embodiments of the invention, less than 5%, such as less than 4%, such as less than 3%, such as less than 2%, such as less than 1%, such as 0.0%, 0.5%, 0.8%, 1.0%, 1.5%, 2.0%, 2.5% of the total molar content of HMOs produced by the cell is LNDFH-III.

In additional embodiments less than 5% of the molar content of the total HMOs produced by the cell is an alternative complex fucosylated HMO. In the present context, an alternative fucosylated HMO is considered one or more fucosylated HMO which is not LNFP-III.

In additional embodiments less than 20%, such as less than 15%, such as less than 10%, such as less than 5%, such as less than 1% of the total molar HMO content of the total HMOs produced by the cell is a non-complex fucosylated HMO, such as 3-FL. The alternative fucosylated HMO(s) may be selected from the group consisting of 3-FL, DFL, LNFP-VI and LNDFH-III. The alternative complex fucosylated HMO may be selected from the groups consisting of LNFP-VI and LNDFH-III.

Production of LNFP-III may require the presence of two or more glycosyltransferase activities, in particular, if starting from lactose as the acceptor oligosaccharide.

### An Acceptor Oligosaccharide

A genetically engineered cell according to the present invention comprises a recombinant nucleic acid sequence encoding a fucosyltransferase with α-1,3-fucosyltransferase activity capable of transferring fucose from an activated sugar to the GlcNAc moiety of an acceptor oligosaccharide, in an α-1,3 linkage.

In the context of the present invention, an acceptor oligosaccharide is an oligosaccharide that can act as a substrate for a glycosyltransferase capable of transferring a glycosyl moiety from a glycosyl donor to the acceptor oligosaccharide. The glycosyl donor is preferably a nucleotide-activated sugar as described in the section on “glycosyltransferases”. Preferably, the acceptor oligosaccharide is a precursor for making a more complex HMO and can also be termed the precursor molecule.

The acceptor oligosaccharide can be either an intermediate product of the present fermentation process, an end-product of a separate fermentation process employing a separate genetically engineered cell, or an enzymatically or chemically produced molecule.

In the present context, said acceptor oligosaccharide for the α-1,3-fucosyltransferase is preferably lacto-N-neotetraose (LNnT), which is produced from the precursor molecules lactose (e.g., acceptor for the β-1,3-N-acetyl-glucosaminyl-transferase) and/or lacto-N-triose II (LNT-II) (e.g., acceptor for the β-1,4-galactosyltransferase). The precursor molecule is preferably fed to the genetically engineered cell which is capable of producing LNnT from the precursor.

### Glycosyltransferases

The genetically engineered cell according to the present invention comprises at least one recombinant nucleic acid sequence encoding at least one glycosyltransferase capable of transferring a fucosyl residue from a fucosyl donor to an acceptor oligosaccharide to synthesize one or more fucosylated human milk oligosaccharide product, i.e., a fucosyltransferase.

The genetically engineered cell according to the present invention may comprise at least one or more further recombinant nucleic acids encoding one or more heterologous glycosyltransferases capable of transferring a glycosyl residue from a glycosyl donor to an acceptor oligosaccharide. Preferably, the additional glycosyltransferase(s) enables the genetically engineered cell to synthesize LNnT from a precursor molecule, such as lactose or LNT-II. In embodiments, the genetically engineered cell of the present invention, comprises one or more further recombinant nucleic acids encoding one or more heterologous glycosyltransferases.

The additional glycosyltransferase is preferably selected from the group consisting of, galactosyltransferases, glucosaminyltransferases, fucosyltransferases and N-acetylglucosaminyl transferases.

In one aspect, the fucosyltransferase in the genetically engineered cell of the present invention is an α-1,3-fucosyltransferase. Preferably, the α-1,3-fucosyltransferase is capable of transferring a fucose unit onto the GlcNAc moiety of an LNnT molecule.

In one embodiment the the α-1,3-fucosyltransferase in the genetically engineered cell primarily fucosylates the N-acetylglucoseamine (GlcNAc) moiety of an acceptor oligosaccharide in an α-1,3 linkage, such as the N-acetylglucoseamine (GlcNAc) moiety LNnT. It is advantageous that the fucosyltransferase has low or no activity on the glucose moiety in the reducing end of an acceptor molecule, such as lactose or an HMO, such as LNT-II or LNnT, in particular LNnT, since the lack of such activity will result in less by-products, e.g., LNFP-VI and/or 3-FL.

In the present invention, the at least one functional enzyme (α-1,3-fucosyltransferase) capable of transferring a fucosyl moiety from a fucosyl donor to an acceptor oligosaccharide can be selected from the group consisting of Camp1, Parm1, Hpull1, Med1 and Hacin2 with an amino acid sequence according to SEQ ID NO: 1, 2, 3, 4 or 5, or a functional homologue thereof with an amino acid sequence that is at least 80% identical to SEQ ID NO: 1, 2, 3, 4 or 5 [Camp1, Parm1, Hpull1, Med1 and Hacin2 amino acid] (table 1). These enzymes can e.g., be used to produce LNFP-III.

In preferred embodiments the α-1,3-fucosyltransferase is selected from the group consisting of Camp1, Parm1 and Hpull1 with an amino acid sequence according to SEQ ID NO: 1, 2, or 3, or a functional homologue thereof with an amino acid sequence that is at least 80% identical to SEQ ID NO: 1, 2, or 3 [Camp1, Parm1 and Hpull1 amino acid] (table 1). These enzymes can e.g., be used to produce LNFP-III.

Without being bound by theory, an α-1,3-fucosyltransferase with a higher substrate-specificity for the GlcNAc moiety in LNnT compared to the substrate-specificity for the terminal glucose moiety in LNnT is advantageous, since such an α-1,3-fucosyltransferase would in theory produce less or no LNFP-VI and 3-FL when the initial substrate is lactose and wherein the availability of LNnT is not limited. A lower amount of LNFP-VI allows for an easier purification of LNFP-III, as the purification of LNFP-III from a mixture of HMOs predominantly comprising LNFP-III would be simpler, as it is easier to separate LNFP-III from smaller HMOs than separating different fucosylated HMOs of the same or similar size from each other, e.g., LNFP-Ill from LNFP-VI or LNDFH-III. Hence, a lower initial amount LNFP-VI and/or LNDFH-III is considered beneficial in the purification of LNFP-III.

In preferred embodiments, the use of an α-1,3-fucosyltransferase according to the present invention results in that at least 90% of the molar content of the total HMOs produced by a cell according to the present invention is LNFP-III. In a further preferred embodiment, the α-1,3-fucosyltransferase is camp1 with an amino acid sequence according to SEQ ID NO: 1 or a functional homologue thereof with an amino acid sequence that is at least 80% identical to SEQ ID NO: 1.

In embodiments, the expression of an α-1,3-fucosyltransferase according to the present invention in a genetically engineered cell is further combined with expression of one or more further recombinant nucleic acids encoding one or more heterologous glycosyltransferases. In preferred embodiments, the expression of an α-1,3-fucosyltransferase of the invention in a genetically engineered cell is combined with expression of a β-1,4-galactosyltransferase, such as galT from Helicobacter pylori. In a further embodiment, a third enzyme is added, such as a β-1,3-N-acetyl-glucosaminyl-transferase, e.g., LgtA from Neisseria meningitidis.

α-1,3-fucosyltransferase

The term “α-1,3-fucosyltransferase” refers to a glycosyltransferase that catalyzes the transfer of fucosyl from a donor substrate, such as GDP-fucose, to an acceptor molecule in an α-1,3-linkage. Preferably, an β-1,3-fucosyltransferase used in the present invention does not originate in the species of the genetically engineered cell, i.e., the gene encoding the α-1,3-fucosyltransferase is of heterologous origin and is selected from an α-1,3-fucosyltransferase identified in table 1. In the context of the present invention, the acceptor molecule for the α-1,3-fucosyltransferase is preferably an acceptor oligosaccharide of at least four monosaccharide units, e.g., LNnT, with a GlcNAc moiety. Heterologous α-1,3-fucosyltransferases that are capable of transferring a fucosyl moiety onto LNnT are known in the art, specifically FutB with an amino acid sequence as provided in SEQ ID NO: 6 and encoded by the nucleic acid sequence of SEQ ID NO: 40, is known to produce a mixture of LNFP-III and LNFP-VI (Dumon et al 2004 Biotechnol. Prog. 20:412-419).

The fucosyltransferase can be selected from an amino acid sequence with at least 80%, such as at least 85%, such as at least 90%, such as at least 95%, or such as at least 99% sequence identity to the amino acid sequence of any one of the α-1,3-fucosyltransferases listed in table 1.

TABLE 1
List of α-1,3-fucosyltransferase enzymes
capable of producing high levels of LNFP-III.

| Enzyme | | SEQ | |
|---|---|---|---|
| Name | GenBank ID¹ | ID NO: | Origin |
| Camp1 | MBP3676691.1 | 1 | Campylobacter sp. |
| Parm1 | WP_129943149.1 | 2 | Parabacteroides merdae |
| Hpull1 | WP_065831246.1 | 3 | Helicobacter pullorum |
| Med1 | WP_087337236.1 | 4 | Mediterranea sp. An20 |
| Hacin2 | WP_104687422.1 | 5 | Helicobacter acinonychis |

¹The GenBank IDs reflect the full-length enzymes, in the present invention truncated or mutated versions may have been used, these are represented by the sequences indicated by the SEQ ID NOs.

Example 1 of the present invention discloses the identification of the heterologous α-1,3-fucosyltransferases Camp1 Parm1, Hpull1, Med1 and Hacin2 (SEQ ID NO: 1, 2, 3, 4 and 5 respectively), which are capable of producing mixtures of HMOs with a higher LNFP-III content when introduced into an LNnT producing cell, than the previously known α-1,3-fucosyltransferase FutB (SEQ ID NO: 6). Specifically, the five novel enzymes Camp1, Parm1, Hpull1, Med1 and Hacin2 can transfer a fucosyl unit onto the GlcNAc moiety of LNnT in an α-1,3 linkage to form LNFP-III at a level above 30% of the total HMO as compared to the prior art enzyme FutB which only produces 6% LNFP-III of the total HMO, and in addition, none of the five novel enzymes produces LNFP-VI.

Furthermore, the experiments performed in Example 1 show that the enzymes Camp1, Hpull1, Med1 and Hacin2 also do not produce any LNDFH-III, indicating that these enzymes are highly substrate-specific for the GlcNAc moiety in LNnT. In contrast, FutB was found to produce minor amounts of LNFP-VI as well as more 3-FL with a single copy of the enzyme compared to the 5 novel enzymes, indicating that FutB also has at least some activity to the terminal glucose (Glc) moiety of LNnT.

In embodiments, the fucosyltransferase with α-1,3-fucosyltransferase activity is selected from the group consisting of Camp1, with an amino acid sequence according to SEQ ID NO: 1 or a functional homologue thereof with an amino acid sequence that is at least 80% identical to SEQ ID NO: 1, Parm1 with an amino acid sequence according to SEQ ID NO: 2 or a functional homologue thereof with an amino acid sequence that is at least 80% identical to SEQ ID NO: 2, Hpull1 with an amino acid sequence according to SEQ ID NO: 3, or a functional homologue thereof with an amino acid sequence that is at least 80% identical to SEQ ID NO: 3, Med1 with an amino acid sequence according to SEQ ID NO: 4, or a functional homologue thereof with an amino acid sequence that is at least 80% identical to SEQ ID NO: 4 and Hacin2 with an amino acid sequence according to SEQ ID NO: 5, or a functional homologue thereof with an amino acid sequence that is at least 80% identical to SEQ ID NO: 5.

In embodiments, the fucosyltransferase with α-1,3-fucosyltransferase activity is selected from the group consisting of Camp1, with an amino acid sequence according to SEQ ID NO: 1 or a functional homologue thereof with an amino acid sequence that is at least 80% identical to SEQ ID NO: 1, Parm1 with an amino acid sequence according to SEQ ID NO: 2 or a functional homologue thereof with an amino acid sequence that is at least 80% identical to SEQ ID NO: 2, and Hpull1 with an amino acid sequence according to SEQ ID NO: 3, or a functional homologue thereof with an amino acid sequence that is at least 80% identical to SEQ ID NO: 3.

In one embodiment of the invention, the fucosyltransferase with α-1,3-fucosyltransferase activity is Camp1 from Campylobacter sp. comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1.

In another embodiment of the invention, the fucosyltransferase with α-1,3-fucosyltransferase activity is Parm1 from Parabacteroides merdae comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2.

In another embodiment of the invention, the fucosyltransferase with α-1,3-fucosyltransferase activity is Hpull1 from Helicobacter pullorum comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3.

In another embodiment of the invention, the fucosyltransferase with α-1,3-fucosyltransferase activity is Med1 from Mediterranea sp. An20 comprising or consisting of the amino acid sequence of SEQ ID NO: 4 or an amino acid sequence with at least 80%, such as at least 85%, such as at least 90%, such as at least 95%, or such as at least 99% sequence identity to SEQ ID NO: 4.

In another embodiment of the invention, the fucosyltransferase with α-1,3-fucosyltransferase activity is Hacin2 from Helicobacter acinonychis comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5.

In one embodiment, the enzyme Camp1 is introduced into a genetically engineered cell which further comprises a β-1,4-galactosyltransferase and preferably also a β-1,3-N-acetyl-glucosaminyl-transferase.

β-1,3-N-acetyl-glucosaminyl-transferase

A β-1,3-N-acetyl-glucosaminyl-transferase is any protein which comprises the ability of transferring the N-acetyl-glucosamine of UDP-N-acetyl-glucosamine to lactose or another acceptor molecule, in a beta-1,3-linkage. Preferably the β-1,3-N-acetyl-glucosaminyl-transferase used herein does not originate in the species of the genetically engineered cell, i.e., the gene encoding the β-1,3-N-acetyl-glucosaminyl-transferase is of heterologous origin. In the context of the present invention, the acceptor molecule is either lactose or an oligosaccharide of at least four monosaccharide units, e.g., LNnT, or more complex HMO structures.

Accordingly, in embodiments, the genetically engineered cell further comprises one or more recombinant nucleic acid sequence(s) encoding a β-1,3-N-acetyl-glucosaminyltransferase.

Non-limiting examples of β-1,3-N-acetyl-glucosaminyltransferases are given in table 2. β-1,3-N-acetyl-glucosaminyltransferase variants may also be useful, preferably such variants are at least 80%, such as at least 85%, such as at least 90%, such as at least 95% identical to the amino acid sequence of any one of the β-1,3-N-acetyl-glucosaminyltransferase in table 2.

TABLE 2
List of β-1,3-N-acetyl-glucosaminyltransferase

| Protein Name | GenBank ID | origin |
|---|---|---|
| lgtA | WP_033911473.1 | Neisseria meningitidis |
| | WP_002248149 | |
| | AAF42258.1 | |
| | ELK60643.1 | |
| lgtA | AAK70338.1 | Neisseria gonorrhoeae |
| | ACF31229.1 | |
| lgtA | AAK02595.1 | Pasteurella multocida |
| lgtA | EEZ72046.1 | Neisseria cinerea |
| HD0466 | WP_010944479.1 | Haemophilus ducreyi |
| PmnagT | WP_014390683.1 | Pasteurella multocida |

In embodiments, the genetically engineered cell comprises a recombinant nucleic acid sequence encoding α-1,3-N-acetyl-glucosaminyltransferase. In one embodiment, the recombinant nucleic acid sequence encoding a β-1,3-N-acetylglucosaminyltransferase comprises or consists of the amino acid sequence of SEQ ID NO: 13 (LgtA from N. meningitidis) or a functional homologue thereof with an amino acid sequence with at least 80%, such as at least 85%, such as at least 90%, such as at least 95%, or such as at least 99% sequence identity to SEQ ID NO: 13.

For the production of LNnT from lactose as substrate, the LNT-II precursor is formed using a β-1,3-N-acetylglucosaminyltransferase. In one embodiment the genetically engineered cell comprises a β-1,3-N-acetylglucosaminyltransferase gene, or a functional homologue or fragment thereof, to produce the intermediate LNT-II from lactose.

Some of the examples below use the heterologous β-1,3-N-acetyl-glucosaminyl-transferase named LgtA from Neisseria meningitidis or a variant thereof.

β-1,4-galactosyltransferase

A β-1,4-galactosyltransferase is any protein that comprises the ability of transferring the galactose of UDP-Galactose to a N-acetyl-glucosaminyl moiety to an acceptor molecule in a beta-1,4-linkage. Preferably, a β-1,4-galactosyltransferase used herein does not originate in the species of the genetically engineered cell i.e., the gene encoding the β-1,4 -galactosyltransferase is of heterologous origin. In the context of the present invention the acceptor molecule, is an acceptor saccharide, e.g., LNT-II, or more complex HMO structures.

The examples below use the heterologous β-1,4-galactosyltransferase named GalT or a variant thereof, to produce e.g., LNFP-III, in combination with other glycosyl transferases. Accordingly, in embodiments, the genetically engineered cell comprises one or more recombinant nucleic acid sequence(s) encoding a β-1,4-galactosyltransferase.

Non-limiting examples of β-1,4-galactosyltransferases are provided in table 3. β-1,4-galactosyltransferases variants may also be useful, preferably such variants are at least 80%, such as at least 85%, such as at least 90, such as at least 95% identical to the amino acid sequence of any one of the β-1,4-galactosyltransferases in table 3.

TABLE 3
List of beta-1,4-glycosyltransferases

| Protein Name | GenBank ID | Origin |
|---|---|---|
| GalT | WP_001262061.1 | Helicobacter pylori |
| LgtB | AAF42257.1 | Neisseria meningitidis MC58 |

In one embodiment, the recombinant nucleic acid sequence encoding a β-1,4-galactosyltransferases comprises or consists of the amino acid sequence of SEQ ID NO: 14 (GalT from H. pylori) or a functional homologue thereof with an amino acid sequence with at least 80%, such as at least 85%, such as at least 90%, such as at least 95%, or such as at least 99% sequence identity to SEQ ID NO: 14.

To produce LNnT form an LNT-II precursor, a β-1,4-galactosyltransferase is needed. In one embodiment, the genetically engineered cell comprises a β-1,4-galactosyltransferase gene, or a functional homologue or fragment thereof. In embodiments, the β-1,3-N-acetylglucosaminyltransferase is from Neisseria meningitidis and the β-1,4-galactosyltransferase is from Helicobacter pylori. In further embodiments, the 1,3-N-acetylglucosaminyltransferase has an amino acid sequence according to SEQ ID NO: 13 [LgtA amino acid], or a functional homologue thereof with an amino acid sequence that is at least 80% identical to SEQ ID NO: 13 and the β-1,4-galactosyltransferase is has an amino acid sequence according to SEQ ID NO: 14 [GalT amino acid], or a functional homologue thereof with an amino acid sequence that is at least 80% identical to SEQ ID NO: 14.

Below are non-limiting examples of genetically modified strains according to the present invention with specific combinations of glycosyl transferases that will lead to production of LST-c using lactose as initial substrate.

In a non-limiting example, LgtA from Neisseria meningitidis is used in combination with galT from Helicobacter pylori and Camp1 from Campylobacter sp. to produce LNFP-III starting from lactose as initial substrate.

In a non-limiting example, LgtA from Neisseria meningitidis is used in combination with galT from Helicobacter pylori and Parm1 from Parabacteroides merdae to produce LNFP-III starting from lactose as initial substrate.

In a non-limiting example, LgtA from Neisseria meningitidis is used in combination with galT from Helicobacter pylori and Hpull1 from Helicobacter pullorum to produce LNFP-III starting from lactose as initial substrate.

In a non-limiting example, LgtA from Neisseria meningitidis is used in combination with galT from Helicobacter pylori and Med1 from Mediterranea sp. An20 to produce LNFP-III starting from lactose as initial substrate.

In a non-limiting example, LgtA from Neisseria meningitidis is used in combination with galT from Helicobacter pylori and Hacin2 from Helicobacter acinonychis to produce LNFP-III starting from lactose as initial substrate.

In a non-limiting example, galT from Helicobacter pylori is used in combination with Camp1 from Campylobacter sp. to produce LNFP-III starting from LNT-II as initial substrate.

In a non-limiting example, galT from Helicobacter pylori is used in combination with Parm1 from Parabacteroides merdae to produce LNFP-III starting from LNT-II as initial substrate.

In a non-limiting example, galT from Helicobacter pylori is used in combination with Hpull1 from Helicobacter pullorum to produce LNFP-III starting from LNT-II as initial substrate.

In a non-limiting example, galT from Helicobacter pylori is used in combination with Med1 from Mediterranea sp. An20 to produce LNFP-III starting from LNT-II as initial substrate

In a non-limiting example, galT from Helicobacter pylori is used in combination with Hacin2 from Helicobacter acinonychis to produce LNFP-III starting from LNT-II as initial substrate.

### Glycosyl-Donor-Nucleotide-Activated Sugar Pathways

When carrying out the method of this invention, preferably a glycosyltransferase mediated glycosylation reaction takes place in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably the following activated sugar nucleotides are involved in the glycosyl transfer: glucose-UDP-GlcNAc, UDP-galactose, UDP-glucose, UDP-N-acetylglucosamine, UDP-N-acetylgalactosamine (GlcNAc) and CMP-N-acetylneuraminic acid.

The genetically engineered cell according to the present invention can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid.

In one embodiment of the current invention, the genetically engineered cell is capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).

The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically engineered cell can utilize salvaged monosaccharides for sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cell.

### Colanic Acid Gene Cluster

For the production of fucosylated HMOs, the de novo GDP-fucose pathway is important to ensure presence of sufficient GDP-fucose. The colanic acid gene cluster of Escherichia coli encodes selected enzymes involved in the de novo synthesis of GDP-fucose (gmd, wcaG, wcaH, wcal, manB, manC), whereas one or several of the genes downstream of GDP-L-fucose such as wcaJ, which are responsible for the production of the extracellular polysaccharide colanic acid, a major oligosaccharide of the bacterial cell wall, can be deleted to prevent conversion of GDP-fucose to colanic acid.

To secure sufficient amounts of GDP-fucose the promoter of the native colanic acid gene cluster may be exchanged with a stronger promoter, generating a recombinant colanic acid gene cluster, to drive additional production of GDP-fucose. Furthermore, an extra copy of the colanic acid gene cluster or selected genes thereof can be introduced in the genetically engineered cells as described in the examples.

In embodiments, the colanic acid gene cluster may be expressed from its native genomic locus. The expression may be actively modulated. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

In relation to the present disclosure, the term “native genomic locus”, in relation to the colanic acid gene cluster, relates to the original and natural position of the gene cluster in the genome of the genetically engineered cell.

The de novo GDP-fucose pathway genes responsible for the formation of GDP-fucose comprises or consists of the following genes:
i) manA which encodes the protein mannose-6 phosphate isomerase (EC 5.3.1.8, UniProt accession nr. P00946), which facilitates the interconversion of fructose 6-phosphate (F6P) and mannose-6-phosphate;
ii) manB which encodes the protein phosphomannomutase (EC 5.4.2.8, UniProt accession nr P24175), which is involved in the biosynthesis of GDP-mannose by catalyzing conversion mannose-6-phosphate into mannose-1-phosphate;
iii) manC which encodes the protein mannose-1-phosphate guanylyltransferase guanylyltransferase (EC: 2.7.7.13, UniProt accession nr P24174), which is involved in the biosynthesis of GDP-mannose through synthesis of GDP-mannose from GTP and α-D-mannose-1-phosphate;
iv) gmd which encodes the protein GDP-mannose-4,6-dehydratase (UniProt accession nr POAC88), which catalyzes the conversion of GDP-mannose to GDP-4-dehydro-6-deoxy-D-mannose;
v) wcaG (fcl) which encodes the protein GDP-L-fucose synthase (EC 1.1.1.271, UniProt accession nr P32055) which catalyses the two-step NADP-dependent conversion of GDP-4-dehydro-6-deoxy-D-mannose to GDP-fucose.

Accordingly, it is preferred that the genetically engineered cell, when producing one or more fucosylated heterologous products, overexpresses either the entire colonic acid gene cluster (e.g. as identified in SEQ ID NO: 12 or a functional variant thereof) and/or one or more genes of the de novo GDP-fucose pathway selected from the group consisting of manA, manB, manC, gmd and wcaG.

### Lactose Permease

Lactose permease is a membrane protein which is a member of the major facilitator superfamily and can be classified as a symporter, which uses the proton gradient towards the cell to transport β-galactosides such as lactose in the same direction into the cell. In oligosaccharide-production, especially in the production of human milk oligosaccharides (HMOs), lactose is often the initial substrate being decorated to produce any HMO of interest in a bioconversion that happens in the cell interior. Thus, in the production of HMOs, there is a desire to be able to import lactose into the cell, e.g., by expression and/or overexpression of a lactose permease such as lacY of E. coli K.-12.

In embodiments, the lactose permease is as shown in SEQ ID NO: 15, or a functional homologue thereof having an amino acid sequence which is at least 80% identical, such as at least 85%, 90% or 95% identical to SEQ ID NO: 15.

In embodiments, the expression of the lactose permease is regulated by a promoter according to the present invention.

β-galactosidase

A host cell suitable for HMO production, e.g., E. coli, may comprise an endogenous β-galactosidase gene or an exogenous β-galactosidase gene, e.g., E. coli comprises an endogenous lacZ gene (e.g., GenBank Accession Number V00296 (GI: 41901)). For the purposes of the invention, when producing an HMO, the genetically engineered cell is genetically manipulated to either not comprise any β-galactosidase gene or to comprise a β-galactosidase gene that is inactivated. The gene may be inactivated by a complete or partial deletion of the corresponding nucleic acid sequence from the bacterial genome, or the gene sequence is mutated in the way that it is not transcribed, or, if transcribed, the transcript is not translated or if translated to a protein (i.e., β-galactosidase), the protein does not have the corresponding enzymatic activity. In this way the HMO-producing bacterium accumulates an increased intracellular lactose pool which is beneficial for the production of HMOs.

### Transporter Proteins

The oligosaccharide product, such as the HMO produced by the cell, can be accumulated both in the intra- and the extracellular matrix. The product can be transported to the supernatant in a passive way, i.e., it diffuses outside across the cell membrane. The more complex HMO products may remain in the cell, which is likely to eventually impair cellular growth, thereby affecting the possible total yield of the product from a single fermentation. The HMO transport can be facilitated by major facilitator superfamily transporter proteins that promote the effluence of sugar derivatives from the cell to the supernatant. The transporter can be present exogenously or endogenously and is overexpressed under the conditions of the fermentation to enhance the export of the oligosaccharide derivative (HMO) produced. The specificity towards the sugar moiety of the product to be secreted can be altered by mutation by means of known recombinant DNA techniques.

Thus, the genetically engineered cell according to the present invention can further comprise a nucleic acid sequence encoding a transporter protein capable of exporting the fucosylated human milk oligosaccharide product or products, such as transporter protein can for example be a member of the major facilitator superfamily transport proteins.

In the resent years, several new and efficient major facilitator superfamily transporter proteins have been identified, each having specificity for different recombinantly produced HMOs and development of recombinant cells expressing said proteins are advantageous for high scale industrial HMO manufacturing, see for example WO2010/142305, WO2017/042382, WO2021/148615, WO2021/148614, WO2021/148611, and WO2021/148620.

Thus, in one or more exemplary embodiments, the genetically engineered cell according to the method described herein further comprises a gene product that acts as an LNFP-III transporter. The gene product that acts as LNFP-III transporter may be encoded by a recombinant nucleic acid sequence that is expressed in the genetically engineered cell. The recombinant nucleic acid sequence encoding the LNFP-III transporter, may be integrated into the genome of the genetically engineered cell, or expressed using a plasmid.

### YjhB

In an embodiment, the genetically modified cell of the disclosure comprises a nucleic acid sequence encoding an efflux transporter protein capable of exporting the the fucosylated oligosaccharide product, in particular, LNFP-III, into the extracellular medium. In the current context, said efflux transporter protein is preferably encoding a putative MFS (major facilitator superfamily) transporter protein, such as the putative metabolite transport protein YjhB, encoded by the gene yjhB from Escherichia coli. More specifically, the disclosure relates to a genetically modified cell optimized to produce an oligosaccharide, in particular LNFP-III, comprising a recombinant nucleic acid encoding a protein having at least 80%, such as at least 85%, such as at least 90% such as at least 95% or 100% sequence identity to the amino acid sequence of the amino acid sequence having UniProt accession ID P39352.

### YhhS

In an embodiment, the genetically modified cell of the disclosure comprises a nucleic acid sequence encoding an efflux transporter protein capable of exporting the the fucosylated oligosaccharide product, in particular, LNFP-III, into the extracellular medium. In the current context, said efflux transporter protein is preferably encoding a putative MFS (major facilitator superfamily) transporter protein, such as the Uncharacterized MFS-type transporter YhhS, encoded by the gene yhhS from Escherichia coli. More specifically, the disclosure relates to a genetically modified cell optimized to produce an oligosaccharide, in particular LNFP-III, comprising a recombinant nucleic acid encoding a protein having at least 80%, such as at least 85%, such as at least 90% such as at least 95% or 100% sequence identity to the amino acid sequence of the amino acid sequence having UniProt accession ID P37621.

### YebQ

In an embodiment, the genetically modified cell of the disclosure comprises a nucleic acid sequence encoding an efflux transporter protein capable of exporting the the fucosylated oligosaccharide product, in particular, LNFP-III, into the extracellular medium. In the current context, said efflux transporter protein is preferably encoding a putative MFS (major facilitator superfamily) transporter protein, such as the uncharacterized transporter YebQ, encoded by the gene yebQ from Escherichia coli. More specifically, the disclosure relates to a genetically modified cell optimized to produce an oligosaccharide, in particular LNFP-III, comprising a recombinant nucleic acid encoding a protein having at least 80%, such as at least 85%, such as at least 90% such as at least 95% or 100% sequence identity to the amino acid sequence of the amino acid sequence having UniProt accession ID P76269.

### The Genetically Engineered Cell

In the present context, the terms “a genetically engineered cell” and “a genetically engineered cell” are used interchangeably. As used herein “a genetically engineered cell” is a host cell whose genetic material has been altered by human intervention using a genetic engineering technique, such a technique is e.g., but not limited to transformation or transfection e.g., with a heterologous polynucleotide sequence, Crisper/Cas editing and/or random mutagenesis. In one embodiment the genetically engineered cell has been transformed or transfected with a recombinant nucleic acid sequence.

The genetic modifications can e.g., be selected from inclusion of glycosyltransferases, and/or metabolic pathway engineering and inclusion of transporters as described in the above sections, which the skilled person will know how to combine into a genetically engineered cell capable of producing one or more fucosylated HMO's.

In one aspect of the invention, the genetically engineered cell comprises a recombinant nucleic acid sequence encoding a fucosyltransferase with α-1,3-fucosyltransferase activity, which is capable of producing at least 30% LNFP-III of the total molar HMO content produced by the cell. In preferred embodiments, the one or more fucosylated HMOs is LNFP-III. In embodiments, the total HMOs produced by said cell is essentially free of LNFP-VI and/or LNDFH-III. In the present invention, essentially free of LNFP-VI and/or LNDFH-III, is to be understood as a content of LNFP-VI and/or LNDFH-III in the total HMO produced by the cell that is less than 1%.

In additional embodiments, the cell of the present invention produces a mixture of HMOs comprising LNFP-III, LNnT, LNDFH-III and/or pLNnH.

In one embodiment the genetically engineered cell capable of producing one or more fucosylated HMO, comprises a recombinant nucleic acid sequence encoding a fucosyltransferase with α-1,3-fucosyltransferase activity, wherein said enzyme is selected from the group consisting of:
a) Camp1, with an amino acid sequence according to SEQ ID NO: 1 or a functional homologue thereof with an amino acid sequence that is at least 80% identical to SEQ ID NO: 1, [Camp1 amino acid],
b) Parm1 with an amino acid sequence according to SEQ ID NO: 2 or a functional homologue thereof with an amino acid sequence that is at least 80% identical to SEQ ID NO: 2, [Parm1 amino acid],
c) Hpull1 with an amino acid sequence according to SEQ ID NO: 3, or a functional homologue thereof with an amino acid sequence that is at least 80% identical to SEQ ID NO: 3 [Hpull1 amino acid],
d) Med1 with an amino acid sequence according to SEQ ID NO: 4, or a functional homologue thereof with an amino acid sequence that is at least 80% identical to SEQ ID NO: 4 [Med1 amino acid] and
e) Hacin2 with an amino acid sequence according to SEQ ID NO: 5, or a functional homologue thereof with an amino acid sequence that is at least 80% identical to SEQ ID NO: 5 [Hacin2 amino acid].

In preferred embodiments the genetically engineered cell capable of producing one or more fucosylated HMO, comprises a recombinant nucleic acid sequence encoding a fucosyltransferase with α-1,3-fucosyltransferase activity, wherein said enzyme is selected from the group consisting of:
a) Camp1, with an amino acid sequence according to SEQ ID NO: 1 or a functional homologue thereof with an amino acid sequence that is at least 80% identical to SEQ ID NO: 1, [Camp1 amino acid],
b) Parm1 with an amino acid sequence according to SEQ ID NO: 2 or a functional homologue thereof with an amino acid sequence that is at least 80% identical to SEQ ID NO: 2, [Parm1 amino acid],
c) Hpull1 with an amino acid sequence according to SEQ ID NO: 3, or a functional homologue thereof with an amino acid sequence that is at least 80% identical to SEQ ID NO: 3 [Hpull1 amino acid] and
d) Med1 with an amino acid sequence according to SEQ ID NO: 4, or a functional homologue thereof with an amino acid sequence that is at least 80% identical to SEQ ID NO: 4 [Med1 amino acid].

In further preferred embodiments the genetically engineered cell capable of producing one or more fucosylated HMO, comprises a recombinant nucleic acid sequence encoding a fucosyltransferase with α-1,3-fucosyltransferase activity, wherein said enzyme is selected from the group consisting of:
a) Camp1, with an amino acid sequence according to SEQ ID NO: 1 or a functional homologue thereof with an amino acid sequence that is at least 80% identical to SEQ ID NO: 1, [Camp1 amino acid],
b) Parm1 with an amino acid sequence according to SEQ ID NO: 2 or a functional homologue thereof with an amino acid sequence that is at least 80% identical to SEQ ID NO: 2, [Parm1 amino acid] and
c) Hpull1 with an amino acid sequence according to SEQ ID NO: 3, or a functional homologue thereof with an amino acid sequence that is at least 80% identical to SEQ ID NO: 3 [Hpull1 amino acid].

In further preferred embodiments the genetically engineered cell capable of producing one or more fucosylated HMO, comprises a recombinant nucleic acid sequence encoding a fucosyltransferase with α-1,3-fucosyltransferase activity, wherein said enzyme is selected from the group consisting of:
a) Camp1, with an amino acid sequence according to SEQ ID NO: 1 or a functional homologue thereof with an amino acid sequence that is at least 80% identical to SEQ ID NO: 1, [Camp1 amino acid] and
b) Parm1 with an amino acid sequence according to SEQ ID NO: 2 or a functional homologue thereof with an amino acid sequence that is at least 80% identical to SEQ ID NO: 2, [Parm1 amino acid].

In further preferred embodiments the genetically engineered cell capable of producing one or more fucosylated HMO, comprises a recombinant nucleic acid sequence encoding a fucosyltransferase with α-1,3-fucosyltransferase activity, wherein said enzyme is selected from the group consisting of:
a) Camp1, with an amino acid sequence according to SEQ ID NO: 1 or a functional homologue thereof with an amino acid sequence that is at least 80% identical to SEQ ID NO: 1, [Camp1 amino acid],
b) Hpull1 with an amino acid sequence according to SEQ ID NO: 3, or a functional homologue thereof with an amino acid sequence that is at least 80% identical to SEQ ID NO: 3 [Hpull1 amino acid].

In a presently preferred embodiments, the genetically engineered cell capable of producing one or more fucosylated HMO, comprises a recombinant nucleic acid sequence encoding a fucosyltransferase with α-1,3-fucosyltransferase activity, wherein said enzyme is Camp1, with an amino acid sequence according to SEQ ID NO: 1 or a functional homologue thereof with an amino acid sequence that is at least 80% identical to SEQ ID NO: 1, [Camp1 amino acid]

In a presently preferred embodiment, the genetically engineered cell capable of producing a fucosylated HMO, which comprises a recombinant nucleic acid sequence encoding a fucosyltransferase with α-1,3-fucosyltransferase activity as described herein is capable of producing LNFP-III in an amount of at least 30%, such as at least 50%, such as at least 80% of the total molar HMO content produced by the cell.

### HMO Mixtures Produced by the Cell

The genetically engineered cell comprising more than two glycosyltransferases described herein will generally produce a mixture of HMOs as a result of the multistep process towards the final HMO product. In the production of LNFP-III from lactose as the initial substrate, it is expected that minor amounts of 3-FL (fucosylated lactose), LNT-II, LNnT, LNFP-III, and pLNnH, and potentially also LNFP-VI and LNDFH-will be produced by the cell.

In some embodiments, the genetically engineered cell of the present invention expresses Camp1 comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1, and the molar % content of LNFP-III produced by the genetically engineered cell is above 90%, such as above 94%, such as above 96%, such as above 97% of the total HMO.

In some embodiments, the genetically engineered cell of the present invention expresses Parm1 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2, and the molar % content of LNFP-Ill produced by the genetically engineered cell is above 70%, such as above 75%, such as above 80%, such as above 85% of the total HMO.

In some embodiments, the genetically engineered cell of the present invention expresses Hpull1 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3, and the molar % content of LNFP-Ill produced by the genetically engineered cell is above 70%, such as above 75%, of the total HMO.

In some embodiments, the genetically engineered cell of the present invention expresses Med1 comprising or consisting of the amino acid sequence of SEQ ID NO: 4 or an amino acid sequence with at least 80%, such as at least 85%, such as at least 90%, such as at least 95%, or such as at least 99% sequence identity to SEQ ID NO: 4, and the molar % content of LNFP-Ill produced by the genetically engineered cell is above 50%, such as above 60%, such as above 70%, such as above 75% of the total HMO.

In some embodiments, the genetically engineered cell of the present invention expresses Hacin2 comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5, and the molar % content of LNFP-Ill produced by the genetically engineered cell is above 30% of the total HMO.

The molar % of individual HMO components supported by experimental data from the Examples shows exemplary HMO composition ranges, wherein the mixture of HMOs consists essentially of LNFP-III and LNnT, 3-FL or pLNnH.

In that regard, a mixture of HMOs may consist essentially of i) LNFP-III and LNnT, ii) LNFP-III and 3-FL, iii) LNFP-III, LNnT and LNDFH-III, or iv) LNFP-III, LNnT and pLNnH.

In some embodiments, the genetically engineered cell of the present invention expresses Camp1 comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1 and the produced mixture consists essentially of LNFP-III and LNnT or LNFP-III and 3-FL, wherein 1-5% of the total molar HMO content in the mixture is LNnT or 1-20% of the total molar HMO content in the mixture is 3-FL and 95-99% of the total molar HMO content in the mixture is LNFP-III, in total adding up to 100% molar content.

In some embodiments, the genetically engineered cell of the present invention expresses Parm1 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2 and the produced mixture consists essentially of LNFP-III, LNnT and LNDFH-III, wherein 3-25% of the total molar HMO content in the mixture is LNnT, 1-10% of the total molar HMO content in the mixture is LNDFH-III, and 70-90% of the total molar HMO content in the mixture is LNFP-III, in total adding up to 100% molar content.

In some embodiments, the genetically engineered cell of the present invention expresses Hpull1 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3 and the produced mixture consists essentially of LNFP-III and LNnT, wherein 10-30% of the total molar HMO content in the mixture is LNnT and 70-90% of the total molar HMO content in the mixture is LNFP-III, in total adding up to 100% molar content.

In some embodiments, the genetically engineered cell of the present invention expresses Med1 comprising or consisting of the amino acid sequence of SEQ ID NO: 4 or an amino acid sequence with at least 80%, such as at least 85%, such as at least 90%, such as at least 95%, or such as at least 99% sequence identity to SEQ ID NO: 4 and the produced mixture consists essentially of LNFP-III and LNnT, or LNFP-III, LNnT and pLNnH or LNFP-III and 3-FL, wherein 0-45% of the total molar HMO content in the mixture is LNnT, 0-8% of the total molar HMO content in the mixture is pLNnH, 0-20% of the total molar HMO content in the mixture is 3-FL, and 50-90% of the total molar HMO content in the mixture is LNFP-III, in total adding up to 100% molar content.

In some embodiments, the genetically engineered cell of the present invention expresses Hacin2 comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5 and the produced mixture consists essentially of LNFP-III, LNnT and pLNnH, wherein 40-70% of the total molar HMO content in the mixture is LNnT, 0-10% of the total molar HMO content in the mixture is pLNnH and 20-60% of the total molar HMO content in the mixture is LNFP-III in total adding up to 100% molar content.

### Host Cells

In embodiments, the engineered cell is a microorganism. The genetically engineered cell is preferably a microbial cell, such as a prokaryotic cell or eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be Erwinia herbicola (Pantoea agglomerans), Citrobacter freundii, Campylobacter sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods described herein, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species for the invention described herein. Also included as part of this invention are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), SporoLactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a heterologous product are e.g., yeast cells, such as Komagataella, Kluyveromyces, Yarrowia, Pichia, Saccaromyces, Schizosaccharomyces or Hansenula or from a filamentous fungus of the genera Aspargillus, Fusarium or Thricoderma.

In one or more exemplary embodiments, the genetically engineered cell is selected from the group consisting of Escherichia sp., Bacillus sp., Lactobacillus sp., Corynebacterium sp. and Campylobacter sp.

In one or more exemplary embodiments, the genetically engineered cell is selected from the group consisting of Escherichia coli, Bacillus subtilis, Lactobacillus lactis, Corynebacterium glutamicum, Yarrowia lipolytica, Pichia pastoris, and Saccharomyces cerevisiae.

In one or more exemplary embodiments, the genetically engineered cell is B. subtilis.

In one or more exemplary embodiments, the genetically engineered cell is S. Cerevisiae or P. pastoris.

In one or more exemplary embodiments, the genetically engineered cell is Escherichia coli.

In one or more exemplary embodiments, the invention relates to a genetically engineered cell, wherein the cell is derived from the E. coli K-12 strain or DE3.

### A Recombinant Nucleic Acid Sequence

The present invention relates to a genetically engineered cell comprising a recombinant nucleic acid sequence encoding a fucosyltransferase with α-1,3-fucosyltransferase activity, such as an enzyme selected from the group consisting of Camp1, Parm1, Hpull1, Med1 and Hacin2, and wherein said cell produces Human Milk Oligosaccharides (HMO). In particular, a fucosylated HMO, and preferably with a molar % content of LNFP-III above, or at least of 30% of the total HMO produced.

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e., a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence or other non-coding regulatory sequences.

The recombinant nucleic acid sequence may in addition be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

The invention also relates to a nucleic acid construct comprising a coding nucleic sequence, i.e. recombinant DNA sequence of a gene of interest, e.g., a fucosyltransferase gene, and a non-coding regulatory DNA sequence, e.g., a promoter DNA sequence, e.g., a recombinant promoter sequence derived from the promoter sequence of the lac operon or the glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. It refers to the functional relationship of a transcriptional regulatory sequence to a transcribed sequence. E.g., a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the invention may be a part of the vector DNA, in another embodiment, the construct it is an expression cassette/cartridge that is integrated in the genome of a host cell.

Accordingly, the term “nucleic acid construct” means an artificially constructed segment of nucleic acids, in particular a DNA segment, which is intended to be inserted into a target cell, e.g., a bacterial cell, to modify expression of a gene of the genome or expression of a gene/coding DNA sequence which may be included in the construct. Thus, in embodiments, the present invention relates to a nucleic acid construct comprising a recombinant nucleic acid sequence encoding a fucosyltransferase, wherein said recombinant nucleic acid sequence is selected from the group consisting of nucleic acid sequences encoding Camp1, Parm1, Hpull1, Med1 and Hacin2, such as a nucleic acid sequence according to SEQ ID NO: 7, 8, 9, 10 or 11, or functional variants thereof.

The genetically engineered cell according to the present invention may also comprise multiple copies of the recombinant nucleic acid sequence encoding a fucosyltransferase. Enhancing the copy number of the fucosyltransferase was shown in Example 1 to change the ratio of the produced HMOs. In specific it was shown that increasing the copy number of Camp1 by introduction of a high copy-number plasmid resulted in a slight production of 3-FL, while abolishing production of LNnT from the strain. Furthermore, increasing the copy number of Parm1 to two genomic copies and further to also include a high copy plasmid (pUC57-Parm1-PglpF-amp), increased the relative amount of LNFP-III produced, combined with a decrease in the amount of LNnT produced. The increase in copy nr of Parm1 also increased the formation of LNDFH-III indicating that increased activity also increased the fucosylation of the Glc moiety in LNnT. In addition, increasing the copy number of Med1 to two genetic copies (stain Med1_2) increased the LNFP-III production, while further increase in expression, through additional expression of Med1 from high copy plasmid resulted in a slight decrease in LNFP-II level while a sudden increase in production of 3-FL was observed. In addition, it was observed for the high expressing Med1 strain that all the LNnT produced by the cell was fucosylated.

Accordingly, the copy number variation may be used in the production to tailor specific HMOs mixtures, in this case a mixture comprising LNFP-III, LNnT and/or 3-FL in different ratios, depending on the need for the specific product.

Accordingly, in embodiments, the genetically engineered cell of the present invention comprises one, two, three or more genomic copies of the recombinant nucleic acid sequence encoding the glycosyltransferase selected from the group consisting of Camp1, Parm1, Hpull1, Med1 and Hacin2 with an amino acid sequence according to SEQ ID NO: 1, 2, 3,4 or 5, or a functional homologue thereof with an amino acid sequence that is at least 80% identical to SEQ ID NO: 1, 2, 3 or 4 [Camp1, Parm1, Hpull1, Med1 and Hacin2 amino acid]. In further embodiments, the recombinant nucleic acid sequence encoding the glycosyltransferase selected from the group consisting of Camp1, Parm1, Hpull1, Med1 and Hacin2 with an amino acid sequence according to SEQ ID NO: 1, 2, 3, 4 or 5, or a functional homologue thereof with an amino acid sequence that is at least 80% identical to SEQ ID NO: 1, 2, 3 or 4 [Camp1,

Parm1, Hpull1, Med1 and Hacin2 amino acid], is encoded on a plasmid. In additional embodiments, the plasmid is a high copy number plasmid, preferably, a pUC57 plasmid.

In further embodiments, the genetically engineered cell according to the present invention comprises one, two, three or more genomic copies and/or a plasmid borne copy of the recombinant nucleic acid sequence encoding the glycosyltransferase selected from the group consisting of, Med1 and Hacin2 with an amino acid sequence according to SEQ ID NO: 1, 2, 3,4 or 5, or a functional homologue thereof with an amino acid sequence that is at least 80% identical to SEQ ID NO: 1, 2, 3 or 4 [Camp1, Parm1, Hpull1, Med1 and Hacin2 amino acid].

One embodiment of the invention relates to a nucleic acid construct comprising a recombinant nucleic acid sequence encoding a fucosyltransferase, wherein said recombinant nucleic acid sequence is selected from the group consisting of a) Camp1 comprising or consisting of the nucleic acid sequences of SEQ ID NO: 7 or an nucleic acid sequence with at least 80%, such as at least 85%, such as at least 90%, such as at least 95%, or such as at least 99% sequence identity to SEQ ID NO: 7; b) Parm1 comprising or consisting the nucleic acid sequences of SEQ ID NO: 8 or an nucleic acid sequence with at least 80%, such as at least 85%, such as at least 90%, such as at least 95%, or such as at least 99% sequence identity to SEQ ID NO: 8; c) Hpull1 comprising or consisting the nucleic acid sequence of SEQ ID NO: 9 or an nucleic acid sequence with at least 80%, such as at least 85%, such as at least 90%, such as at least 95%, or such as at least 99% sequence identity to SEQ ID NO: 9, d) Med1 comprising or consisting the nucleic acid sequence of SEQ ID NO: 10 or an nucleic acid sequence with at least 80%, such as at least 85%, such as at least 90%, such as at least 95%, or such as at least 99% sequence identity to SEQ ID NO: 10; and/or e) Hacin2 comprising or consisting the nucleic acid sequence of SEQ ID NO: 11 or an nucleic acid sequence with at least 80%, such as at least 85%, such as at least 90%, such as at least 95%, or such as at least 99% sequence identity to SEQ ID NO: 11.

Preferably, the fucosyltransferase encoding sequence is under the control of a promoter sequence selected from promotor sequences with a nucleic acid sequence as identified in Table 4.

TABLE 4
Selected promoter sequences

| | % Activity | | | Seq ID |
|---|---|---|---|---|
| Promoter name | relative to PglpF* | Strength | Reference | in appl. |
| PmglB_70UTR_SD8 | 291% | high | WO2020255054 | 16 |
| PmglB_70UTR_SD10 | 233-281% | high | WO2020255054 | 17 |
| PmglB_54UTR | 197% | high | WO2020255054 | 18 |
| Plac_70UTR | 182-220% | high | WO2019123324 | 19 |
| PmglB_70UTR_SD9 | 180-226% | high | WO2020255054 | 20 |
| PmglB_70UTR_SD4 | 153%-353% | high | WO2020255054 | 21 |
| PmglB_70UTR_SD5 | 146-152% | high | WO2020255054 | 22 |
| PglpF_SD4 | 140-161% | high | WO2019123324 | 23 |
| PmglB_70UTR_SD7 | 127-173% | high | WO2019123324 | 24 |
| PmglB_70UTR | 124-234% | high | WO2020255054 | 25 |
| PglpA_70UTR | 102-179% | high | WO2019123324 | 26 |
| PglpT_70UTR | 102-240% | high | WO2019123324 | 27 |
| PglpF | 100% | high | WO2019123324 | 28 |
| PglpF_SD10 | 88-96% | high | WO2019123324 | 29 |
| PglpF_SD5 | 82-91% | high | WO2019123324 | 30 |
| PglpF_SD8 | 81-82% | high | WO2019123324 | 31 |
| PmglB_16UTR | 78-171% | high | WO2019123324 | 32 |
| PglpF_SD9 | 73-93% | middle | WO2019123324 | 33 |
| PglpF_SD7 | 47-57% | middle | WO2019123324 | 34 |
| PglpF_SD6 | 46-47% | middle | WO2019123324 | 35 |
| PglpA_16UTR | 38-64% | middle | WO2019123324 | 36 |
| Plac_wt* | 15-28% | low | WO2019123324 | 37 |
| PglpF_SD3 | 9% | low | WO2019123324 | 38 |
| PglpF_SD1 | 5% | low | WO2019123324 | 39 |

*The promoter activity is assessed in the LacZ assay described below with the PglpF promoter run as positive reference in the same assay. To compare across assays the activity is calculated relative to the PglpF promoter, a range indicates results from multiple assays.

The promoter may be of heterologous origin, native to the genetically engineered cell or it may be a recombinant promoter, combining heterologous and/or native elements.

One way to increase the production of a product may be to regulate the production of the desired enzyme activity used to produce the product, such as the glycosyltransferases or enzymes involved in the biosynthetic pathway of the glycosyl donor.

Increasing the promoter strength driving the expression of the desired enzyme may be one way of doing this. The strength of a promoter can be assessed using a lacZ enzyme assay where β-galactosidase activity is assayed as described previously (see e.g., Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU. IN preferred embodiments, the expression of said nucleic acid sequences are under control of a strong promoter selected from the group consisting of SEQ ID NOs 16, 17, 18, 19, 20, 21, 22, 23, 24, 25, 26 and 27.

In embodiments the expression of said nucleic acid sequences of the present invention is under control of a PglpF (SEQ ID NO: 28) or Plac (SEQ ID NO: 37) promoter or PmglB_UTR70 (SEQ ID NO: 25) or PglpA_70UTR (SEQ ID NO: 26) or PglpT_70UTR (SEQ ID NO: 27) or variants thereof such as promoters identified in Table 4, in particular the PglpF_SD4 variant of SEQ ID NO: 23 or Plac_70UTR variant of SEQ ID NO: 19, or PmglB_70UTR variants of SEQ ID NO: 16, 17, 18, 20, 21, 22, 24 and 25. Further suitable variants of PglpF, PglpA_70UTR, PglpT_70UTR and PmglB_70UTR promoter sequences are described in or WO2019/123324 and WO2020/255054 respectively (hereby incorporated by reference).

In preferred embodiments, the recombinant nucleic acid sequences individually are under the control of one or more promoters selected from the group consisting of PglpF, Plac, PmglB_70UTR, PglpA_70UTR and PglpT_70UTR (SEQ ID NOs: 28, 37, 25, 26 and 27, respectively) and variants thereof.

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2 (2): 137-49); methods for genomic integration of nucleic acid sequences in which recombination is mediated by the Red recombinase function of the phage λ or the RecE/RecT recombinase function of the Rac prophage (Murphy, J Bacteriol. (1998); 180 (8): 2063-7; Zhang et al., Nature Genetics (1998) 20:123-128 Muyrers et al., EMBO Rep. (2000) 1 (3): 239-243); methods based on Red/ET recombination (Wenzel et al., Chem Biol. (2005), 12 (3): 349-56; Vetcher et al., Appl Environ Microbiol. (2005); 71 (4): 1829-35); or positive clones, i.e., clones that carry the expression cassette, can be selected e.g., by means of a marker gene, or loss or gain of gene function.

In one or more exemplary embodiments, the present disclosure relates to one or more recombinant nucleic acid sequences as illustrated in SEQ ID NOs: 7, 8, 9, 10 and 11 [nucleic acid encoding Camp1, Parm1, Hpull1, Med1 and Hacin1, respectively].

In particular, the present disclosure relates to one or more of a recombinant nucleic acid sequence and/or to a functional homologue thereof having a sequence which is at least 70% identical to SEQ ID NOs: 7, 8, 9, 10 and 11 [nucleic acid encoding Camp1, Parm1, Hpull1, Med1 and Hacin1, respectively], such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence of the invention) and a reference sequence (such as a prior art sequence) based on their pairwise alignment. For purposes of the present invention, the sequence identity between two amino acid sequences is determined using the Needleman-Wunsch algorithm (Needleman and Wunsch, 1970, J. Mo/. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “identity” (obtained using the—nobrief option) is used as the percent identity. Generally sequence identity may be calculated as follows: (Identical Residues×100)/(Length of Aligned region).

For purposes of the present invention, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16:276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “identity” (obtained using the -nobrief option) is used as the percent identity. Generally sequence identity may be calculated as follows: (Identical Deoxyribonucleotides×100)/(Length of Aligned region).

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences shown in table 1 or a recombinant nucleic acid encoding any one of the sequences of SEQ ID NO: 7, 8, 9, 10 or 11, should ideally be able to participate in the production of fucosylated HMOs, in terms of increased HMO yield, export of HMO product out of the cell or import of substrate for the HMO production, such as a acceptor oligosaccharide of at least three monosaccharide units, improved purity/by-product formation, reduction in biomass formation, viability of the genetically engineered cell, robustness of the genetically engineered cell according to the disclosure, or reduction in consumables needed for the production.

### Use of a Genetically Engineered Cell or Enzyme

The disclosure also relates to any commercial use of the enzyme(s), genetically engineered cell(s) or the nucleic acid construct(s) disclosed herein, such as, but not limited to, in a method for producing one or more fucosylated human milk oligosaccharide (HMO).

Accordingly, the present invention also relates to the use of a fucosyltransferase with α-1,3-fucosyltransferase activity in production of a fucosylated product, wherein the fucosyltransferase is selected from the group consisting of Camp1, Parm1, Hpull1, Med1 and Hacin2 with an amino acid sequence according to SEQ ID NO: 1, 2, 3, 4 or 5, or a functional homologue thereof with an amino acid sequence that is at least 80% identical to SEQ ID NO: 1, 2, 3, 4 or 5 [Camp1, Parm1, Hpull1, Med1 and Hacin2 amino acid]. In further embodiments, the fucosyltransferase for use in production of a fucosylated product is selected from the group consisting of Camp1, Parm1, Hpull1 and Hacin2 with an amino acid sequence according to SEQ ID NO: 1, 2, 3 or 5, or a functional homologue thereof which amino acid sequence is at least 80% identical to SEQ ID NO: 1, 2, 3 or 5 [Camp1, Parm1, Hpull1 and Hacin2 amino acid]. In embodiments, the fucosyltransferase of the present invention are also used in the manufacturing of a fucosylated product, wherein the fucosylated product is one or more fucosylated oligosaccharides, such as one or more HMOs, preferably, LNFP-III, or one or more fucosylated polypeptides.

In an exemplified embodiment, the genetically engineered cell and/or the nucleic acid construct according to the invention is used in the manufacturing of HMOs. Preferably, in the manufacturing of mixtures of HMOs, wherein the molar % content of LNFP-III produced by the genetically engineered cell is above 30% of the total amount of HMO produced. Preferably, in the manufacturing of HMOs, wherein pure LNFP-III is intended as the primary product, the molar % content of LNFP-III produced by the genetically engineered cell is above 70% such as above 75%, such as above 80%, such as above 90% of the total amount of HMO produced.

In further embodiments, the fucosyltransferase for use in production of a fucosylated product is selected from the group consisting of Camp1, Parm1, Hpull1 with an amino acid sequence according to SEQ ID NO: 1, 2 or 3, or a functional homologue thereof which amino acid sequence is at least 80% identical to SEQ ID NO: 1, 2 or 3 [Camp1, Parm1 and Hpull1]. In embodiments, the fucosyltransferase of the present invention are also used in the manufacturing of a fucosylated product, wherein the fucosylated product is one or more fucosylated oligosaccharides, such as one or more HMOs, preferably, LNFP-III, or one or more fucosylated polypeptides, wherein the molar % content of LNFP-III produced by the genetically engineered cell is above 75%.

In an exemplified embodiment, the genetically engineered cell and/or the nucleic acid construct according to the invention is used in the manufacturing of one or more fucosylated HMO(s), preferably, LNFP-III.

Production of these HMO's may require the presence of two or more glycosyltransferase activities.

### a Method for Producing Fucosylated Human Milk Oligosaccharides (HMOs)

The present invention also relates to a method for producing one or more fucosylated human milk oligosaccharide (HMO), said method comprises culturing a genetically engineered cell according to the present invention.

One aspect is a method for producing one or more fucosylated human milk oligosaccharides (HMOs), said method comprises comprising the steps of:
a) providing a genetically modified cell as described herein; and
b) culturing the cell according to (a) in a suitable cell culture medium to produce said one or more fucosylated HMOs, and
c) optionally, purifying said one or more fucosylated HMOs.

In one embodiment of the invention, following cultivation of the genetically engineered cell as described herein, the mixture of HMOs consists essentially of LNFP-III and LNnT with low amounts of 3-FL, LNDFH-III and/or pLNnH, such as below 10% total molar HMO content in the composition.

In one embodiment of the invention, following cultivation of the genetically engineered cell as described herein, the mixture of HMOs consists essentially of 30-55 molar % LNFP-III, 40-65 molar % LNnT and 3-8% pLNnH, in total adding up to 100% molar content.

In one embodiment of the invention, following cultivation of the genetically engineered cell as described herein, the mixture of HMOs consists essentially of 30-99 molar % LNFP-III, 0-65 molar % LNnT, 0-20% 3-FL, 0-10% LNDFH-III and 0-8% pLNnH, in total adding up to 100% molar content

In one embodiment of the invention, following cultivation of the genetically engineered cell as described herein, the mixture of HMOs consists essentially of 70-99% LNFP-III and 1-50% LNnT in total adding up to 100% molar content.

In one embodiment of the invention, following cultivation of the genetically engineered cell as described herein, the mixture of HMOs consists essentially of 80-99% LNFP-III, 0-5% LNnT and 0-10% LNDFH-III, in total adding up to 100% molar content.

In one embodiment of the invention, following cultivation of the genetically engineered cell as described herein, the mixture of HMOs consists essentially of 80-99 molar % LNFP-III and 0-20 molar % 3-FL.

The present invention in particular relates to a method for producing human milk oligosaccharides (HMOs), wherein the molar % content of LNFP-III produced by the genetically engineered cell is above 30% of the total amount of HMO produced, such as above 50% of the total amount of HMO or such as above 80% of the total amount of HMO. In embodiments, the fucosylated HMO is LNFP-III.

The present invention thus relates to a method for producing one or more fucosylated human milk oligosaccharide (HMO), said method comprising culturing a genetically engineered cell, said cell comprising:
a recombinant nucleic acid sequence encoding a fucosyltransferase with α-1,3-fucosyltransferase activity, wherein said enzyme is selected from the group consisting of:
   a. Camp1, with an amino acid sequence according to SEQ ID NO: 1 or a functional homologue thereof with an amino acid sequence that is at least 80% identical to SEQ ID NO: 1, [Camp1 amino acid];
   b. Parm1 with an amino acid sequence according to SEQ ID NO: 2 or a functional homologue thereof with an amino acid sequence that is at least 80% identical to SEQ ID NO: 2, [Parm1 amino acid];
   c. Hpull1 with an amino acid sequence according to SEQ ID NO: 3, or a functional homologue thereof with an amino acid sequence that is at least 80% identical to SEQ ID NO: 3 [Hpull1 amino acid];
   d. Med1 with an amino acid sequence according to SEQ ID NO: 4, or a functional homologue thereof with an amino acid sequence that is at least 80% identical to SEQ ID NO: 4 [Med1 amino acid];
   e. Hacin2 with an amino acid sequence according to SEQ ID NO: 5, or a functional homologue thereof with an amino acid sequence that is at least 80% identical to SEQ ID NO: 5 [Hacin2 amino acid]; and wherein said cell produces a fucosylated HMO, preferably, LNFP-III.

A further embodiment of the invention is a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,4-galactosyltransferase activity; and
c. a recombinant nucleic acid sequence encoding a fucosyltransferase with α-1,3-fucosyltransferase activity, wherein said enzyme is selected from the group consisting of:
   i. Camp1, with an amino acid sequence according to SEQ ID NO: 1 or a functional homologue thereof with an amino acid sequence that is at least 80% identical to SEQ ID NO: 1, [Camp1 amino acid];
   ii. Parm1 with an amino acid sequence according to SEQ ID NO: 2 or a functional homologue thereof with an amino acid sequence that is at least 80% identical to SEQ ID NO: 2, [Parm1 amino acid];
   iii. Hpull1 with an amino acid sequence according to SEQ ID NO: 3, or a functional homologue thereof with an amino acid sequence that is at least 80% identical to SEQ ID NO: 3 [Hpull1 amino acid] and
   iv. Med1 with an amino acid sequence according to SEQ ID NO: 4, or a functional homologue thereof with an amino acid sequence that is at least 80% identical to SEQ ID NO: 4 [Med1 amino acid];
   v. Hacin2 with an amino acid sequence according to SEQ ID NO: 5, or a functional homologue thereof with an amino acid sequence that is at least 80% identical to SEQ ID NO: 5 [Hacin2 amino acid]; and
   wherein at least one of the fucosylated HMOs is LNFP-III.

A further embodiment of the invention relates to a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,4-galactosyltransferase activity; and
c. a recombinant nucleic acid sequence encoding a fucosyltransferase with α-1,3-fucosyltransferase activity, wherein said enzyme is selected from the group consisting of:
   i. Camp1, with an amino acid sequence according to SEQ ID NO: 1 or a functional homologue thereof with an amino acid sequence that is at least 80% identical to SEQ ID NO: 1, [Camp1 amino acid],
   ii. Parm1 with an amino acid sequence according to SEQ ID NO: 2 or a functional homologue thereof with an amino acid sequence that is at least 80% identical to SEQ ID NO: 2, [Parm1 amino acid] and
   iii. Hpull1 with an amino acid sequence according to SEQ ID NO: 3, or a functional homologue thereof with an amino acid sequence that is at least 80% identical to SEQ ID NO: 3 [Hpull1 amino acid].

In embodiments, the fucosylated HMOs is LNFP-III.

The method particularly comprises culturing a genetically engineered cell that produces a fucosylated HMO, wherein the LNFP-III content produced by said cell is at least 30% of the total HMO content produced by the cell. In addition, the method comprises culturing a genetically engineered cell that produces a fucosylated HMO.

The method comprising culturing a genetically engineered cell that produces a fucosylated HMO and further comprises culturing said genetically engineered cell in in the presence of an energy source (carbon source) selected from the group consisting of glucose, sucrose, fructose, xylose and glycerol.

In one aspect, the method according to the present invention produces a mixture of HMO(s), wherein at least 50%, such as at least 55%, 60%, 65%, 70%, 75%, 80%, 85%, 90%, 95% or 98% of the molar content of the total amount of HMOs produced is LNFP-III.

In one aspect, the method according to the present invention produces a mixture of HMO(s), wherein the produced mixture of HMOs is essentially free of LNFP-VI and/or LNDFH-III.

In one aspect, the method according to the present invention produces a mixture of HMO(s), wherein the cell produces a mixture of HMOs comprising LNFP-III, LNnT, 3-FL and/or pLNnH.

In one aspect, the method according to the present invention produces LNFP-III.

To enable the production of fucosylated HMOs in the method according to the present invention, the genetically engineered cell comprises a biosynthetic pathway for making a fucose sugar nucleotide e.g., GDP-fucose.

In preferred embodiments of the methods of the present invention, the genetically engineered cell comprises an upregulated biosynthetic pathway for making a fucose sugar nucleotide. Preferably, in methods of the present invention, the fucose sugar nucleotide is GDP-Fucose. Thus, in methods of the present invention the sugar nucleotide pathway is expressed and/or upregulated in the genetically engineered cell, wherein the GDP-fucose pathway is encoded by the colanic acid gene cluster (CA) from E. coli of SEQ ID NO: 12. In methods of the present invention, the upregulation of the GDP-fucose pathway is obtained by integration of one or more copies of the colanic acid gene cluster (CA) from E. coli of SEQ ID NO: 12 into the genome of the host cell.

The method of the present invention comprises providing a glycosyl donor, which is synthesized separately by one or more genetically engineered cells and/or is exogenously added to the culture medium from an alternative source.

In one aspect, the method of the present invention further comprises providing an acceptor saccharide as substrate for the HMO formation, the acceptor saccharide comprising at least two monosaccharide units, which is exogenously added to the culture medium and/or has been produced by a separate microbial fermentation.

In one aspect, the method of the present invention comprises providing an acceptor saccharide comprising at least two monosaccharide units, which is exogenously added to the culture medium and/or has been produced by a separate microbial fermentation and which is selected form lactose, LNT-II and LNnT. In a preferred embodiment the substrate for HMO formation is lactose which is fed to the culture during the fermentation of the genetically engineered cell.

The fucosylated human milk oligosaccharide (HMO) is retrieved from the culture, either from the culture medium and/or the genetically engineered cell.

A further embodiment of the invention is a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,4-galactosyltransferase activity; and
c. a recombinant nucleic acid sequence encoding a fucosyltransferase with α-1,3-fucosyltransferase activity, wherein said enzyme is Camp1, with an amino acid sequence according to SEQ ID NO: 1 or a functional homologue thereof with an amino acid sequence that is at least 80% identical to SEQ ID NO: 1, [Camp1 amino acid], and
   wherein at least one of the fucosylated HMOs is LNFP-III.

A further embodiment of the invention is a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,4-galactosyltransferase activity; and
c. a recombinant nucleic acid sequence encoding a fucosyltransferase with α-1,3-fucosyltransferase activity, wherein said enzyme is Parm1, with an amino acid sequence according to SEQ ID NO: 2 or a functional homologue thereof with an amino acid sequence that is at least 80% identical to SEQ ID NO: 2, [Parm1 amino acid], and
   wherein at least one of the fucosylated HMOs is LNFP-III.

A further embodiment of the invention is a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,4-galactosyltransferase activity; and
c. a recombinant nucleic acid sequence encoding a fucosyltransferase with α-1,3-fucosyltransferase activity, wherein said enzyme is Hpull1, with an amino acid sequence according to SEQ ID NO: 3 or a functional homologue thereof with an amino acid sequence that is at least 80% identical to SEQ ID NO: 3, [Hpull amino acid], and
   wherein at least one of the fucosylated HMOs is LNFP-III.

A further embodiment of the invention is a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,4-galactosyltransferase activity; and
c. a recombinant nucleic acid sequence encoding a fucosyltransferase with α-1,3-fucosyltransferase activity, wherein said enzyme is Med1, with an amino acid sequence according to SEQ ID NO: 4 or a functional homologue thereof with an amino acid sequence that is at least 80% identical to SEQ ID NO: 4, [Med1 amino acid], and
   wherein at least one of the fucosylated HMOs is LNFP-III.

A further embodiment of the invention is a method for producing one or more fucosylated human milk oligosaccharides (HMO), said method comprising culturing a genetically engineered cell comprising
a. a recombinant nucleic acid sequence encoding an enzyme with β-1,3-N-acetyl-glucosaminyltransferase activity; and
b. a recombinant nucleic acid sequence encoding an enzyme with a β-1,4-galactosyltransferase activity; and
c. a recombinant nucleic acid sequence encoding a fucosyltransferase with α-1,3-fucosyltransferase activity, wherein said enzyme is Hacin2, with an amino acid sequence according to SEQ ID NO: 5 or a functional homologue thereof with an amino acid sequence that is at least 80% identical to SEQ ID NO: 5, [Hacin2 amino acid], and
   wherein at least one of the fucosylated HMOs is LNFP-III.

Culturing or fermenting (used interchangeably herein) in a controlled bioreactor typically comprises (a) a first phase of exponential cell growth in a culture medium ensured by a carbon-source, and (b) a second phase of cell growth in a culture medium run under carbon limitation, where the carbon-source is added continuously together with the acceptor oligosaccharide, such as lactose, allowing formation of the HMO product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

The terms “manufacturing” or “manufacturing scale” or “large-scale production” or “large-scale fermentation”, are used interchangeably and in the meaning of the invention defines a fermentation with a minimum volume of 100 L, such as 1000L, such as 10.000L, such as 100.000L, such as 200.000L culture broth. Usually, a “manufacturing scale” process is defined by being capable of processing large volumes yielding amounts of the HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behaviour of an expression system in a lab scale method, such as shake flasks, benchtop bioreactors or the deep well format described in the examples of the disclosure, does allow to predict the behaviour of that system in the complex environment of a bioreactor.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon-source can be selected from the group consisting of glucose, sucrose, fructose, xylose and glycerol. In one or more exemplary embodiments, the culturing media is supplemented with one or more energy and carbon sources selected form the group containing glycerol, sucrose and glucose. In additional embodiments, lactose is added during the cultivation of the genetically engineered cells as a substrate for the HMO formation.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons as described in WO2015/197082 (hereby incorporated by reference).

After carrying out the method of this invention, the fucosylated HMO produced can be collected from the cell culture or fermentation broth in a conventional manner.

### Retrieving/Harvesting

The fucosylated human milk oligosaccharide (HMO) is retrieved from the culture medium and/or the genetically engineered cell. In the present context, the term “retrieving” is used interchangeably with the term “harvesting”. Both “retrieving” and “harvesting” in the context relate to collecting the produced HMO(s) from the culture/broth following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation broth) include extraction thereof from the biomass (i.e., the production cells).

After recovery from fermentation, HMO(s) are available for further processing and purification.

The HMOs can be purified according to the procedures known in the art, e.g., such as described in WO2017/152918, WO2017/182965 or WO2015/188834, wherein the latter describes purification of fucosylated HMOs. The purified HMOs can be used as nutraceuticals, pharmaceuticals, or for any other purpose, e.g., for research.

At the end of culturing, the oligosaccharide as product can be accumulated both in the intra- and the extracellular matrix.

The method according to the present invention comprises cultivating the genetically engineered microbial cell in a culture medium which is designed to support the growth of microorganisms, and which contains one or more carbohydrate sources or just carbon-source, such as selected from the group consisting of glucose, sucrose, fructose, xylose and glycerol. In one or more exemplary embodiments, the culturing media is supplemented with one or more energy and carbon sources selected form the group containing glycerol, sucrose and glucose.

### Manufactured Product

The term “manufactured product” according to the use of the genetically engineered cell or the nucleic acid construct refer to the one or more HMOs intended as the one or more product HMO(s). The various products are described above.

From the data presented in example 2, it can be seen that the Camp1 fucosyltransferase produced LNFP-III is of very high purity showing the ability and suitability of Camp1 to produce highly pure LNFP-III in large scale manufacturing.

Advantageously, the methods disclosed herein provide both a decreased ratio of by-product to product and an increased overall yield of the product (and/or HMOs in total). This, less by-product formation in relation to product formation, facilitates an elevated product production and increases efficiency of both the production and product recovery process, providing superior manufacturing procedure of HMOs.

The manufactured product may be a powder, a composition, a suspension, or a gel comprising one or more HMOs.

### Mixtures of HMOs

LNFP-III is a very well-studied HMO that has been linked to many different effects including hepatosteatosis and insulin resistance, as well as in autoimmunity in a murine model of Type 1 Diabetes (T1D), and transplantation (Triantis et al., Immunological Effects of Human Milk Oligosaccharides, Front Pediatr. 2018 Jul. 2; 6:190 eCollection 2018).

Accordingly, LNFP-III and mixtures thereof are highly relevant as either a nutritional supplement or as a therapeutic.

Thus, the manufactured product may be a mixture of HMOs consisting essentially of i) LNFPIII and LNnT, ii) LNFP-III and 3-FL, iii) LNFP-III LNnT and pLNnH, or iv) LNFP-III LNnT and LNDFH-III and wherein further the composition is essentially free of LNFP-VI.

In addition, the manufactured product may be a mixture of HMOs consisting essentially of i) LNFPIII and LNnT, ii) LNFP-III and 3-FL and iii) LNFP-III LNnT and pLNnH and wherein further the composition is essentially free of LNFP-VI and LNDFH-III.

In that regard, in embodiments, a mixture of HMOs according to the present invention consists essentially of 30-55 molar % LNFP-III, 40-65 molar % LNnT and 3-8 molar % pLNnH, in total adding up to 100 molar % molar content.

In additional embodiments, the mixture of HMOs according to the present invention consists essentially of 30-99 molar % LNFP-III, 0-65 molar % LNnT, 0-20 molar % 3-FL, 0-10 molar % LNDFH-III and 0-8 molar % pLNnH, in total adding up to 100% molar content

In other embodiments, the mixture of HMOs according to the present invention consists essentially of 70-99 molar % LNFP-III and 1-50 molar % LNnT in total adding up to 100% molar content. In other embodiments, the mixture of HMOs according to the present invention consists essentially of 80-99 molar % LNFP-III, 0-5 molar % LNnT and 0-10 molar % LNDFH-III, in total adding up to 100% molar content.

In other embodiments, the mixture of HMOs according to the present invention consists essentially of 80-99 molar % LNFP-III and 0-20 molar % 3-FL, in total adding up to 100% molar content.

In embodiments, the present invention also relates to a composition comprising a mixture of HMOs, wherein the composition comprises 30-55 molar % LNFP-III, 40-65 molar % LNnT and 3-8 molar % pLNnH of the total molar HMO content in the composition, in total adding up to 100% molar content. A preferred embodiment is a mixture consisting essentially of 45 to 55 molar % LNFP-III and 45 to 55 molar % LNnT of the total molar HMO content in the composition, in total adding up to 100% molar content. In another embodiment is a mixture consisting essentially of 35 to 45 molar % LNFP-III and 50 to 60 molar % LNnT and 4 to 6 molar % pLNnH of the total molar HMO content in the composition, in total adding up to 100% molar content.

In further embodiments, the present invention also relates to a composition comprising a mixture of HMOs, wherein the composition comprises 30-99 molar % LNFP-III, 0-65 molar % LNnT, 0-20 molar % 3-FL, 0-10 molar % LNDFH-III and 0-8 molar % pLNnH of the total molar HMO content in the composition, in total adding up to 100% molar content. A preferred embodiment is a mixture consisting essentially of 50 to 85 molar % LNFP-III and 15 to 50 molar % LNnT of the total molar HMO content in the composition, in total adding up to 100% molar content. Another preferred embodiment is a mixture consisting essentially of 60 to 85 molar % LNFP-III and 15 to 40 molar % LNnT of the total molar HMO content in the composition, in total adding up to 100% molar content

In other embodiments, the mixture of HMOs according to the present invention consists essentially of 50 molar % LNFP-III, and 50 molar % LNnT.

In other embodiments, the mixture of HMOs according to the present invention consists essentially of 75 molar % LNFP-III, and 25 molar % LNnT.

In other embodiments, the mixture of HMOs according to the present invention consists essentially of 40 molar % LNFP-III, and 55 molar % LNnT and 5 molar % pLNnH.

In additional embodiments, the present invention also relates to a composition comprising a mixture of HMOs, wherein the composition comprises 70-99 molar % LNFP-III and 1-50 molar % LNnT of the total molar HMO content of the composition, in total adding up to 100% molar content.

In additional embodiments, the present invention also relates to a composition comprising a mixture of HMOs, wherein the composition comprises 80-99 molar % LNFP-III, 0-5 molar % LNnT and 0-10 molar % LNDFH-III of the total molar HMO content of the composition, in total adding up to 100% molar content.

In additional embodiments, the present invention also relates to a composition comprising a mixture of HMOs, wherein the composition comprises 80-99 molar % LNFP-III and 0-20 molar % 3-FL of the total molar HMO content of the composition, in total adding up to 100% molar content.

Clinical data in infants indicate that Human Milk Oligosaccharide supplements may help to develop the desired microbiota by serving as a food source for the good bacteria in the intestine. Naturally occurring in breast milk, HMOs have evolved over thousands of years, with HMO research (clinical and preclinical) now suggesting that specific HMOs at the correct level of supplementation can provide us with unique health benefits. In particular, Human Milk Oligosaccharide supplements may help support immunity and gut health, with a potential role in cognitive development, which may open future innovation opportunities.

Mixtures of HMOs may also form part of a composition comprising additional parts, such as active pharmaceutical ingredients, food supplements, excipients, surfactants etc.

Accordingly, the present invention also relates to the use of a composition comprising a mixture of HMOs, wherein the composition comprises LNFPIII and LNnT, LNFP-III and 3-FL, LNFP-III LNnT and pLNnH or LNFP-III, LNnT and 3-FL and wherein further the composition is essentially free of LNFP-VI and has less than 5% LNDFH-III, in an infant formula, a dietary supplement or medical nutrition.

In embodiments, the composition comprising a mixture of HMOs is a pharmaceutical composition.

### Use of HMO Mixtures and Compositions

Clinical data in infants, indicate that Human Milk Oligosaccharide supplements may help to develop the desired microbiota by serving as a food source for the good bacteria in the intestine. Naturally occurring in breast milk, HMOs have evolved over thousands of years, with HMO research (clinical and preclinical) now suggesting that specific HMO's at the correct level of supplementation can provide us with unique health benefits. In particular, Human Milk Oligosaccharide supplements may help support immunity and gut health including a support of a balanced microbiome, with a potential role in cognitive development, which may open future innovation opportunities.

Accordingly, in embodiments, the invention relates to the use of a mixture or composition according to the present invention in infant nutrition.

The present invention also relates to the use of a mixture or composition according to the present invention as a dietary supplement or medical nutrition.

The mixtures or composition of HMOs produced according to the method described herein may be used to enhance the beneficial bacteria in the gut microbiome. Beneficial bacteria are for example bacteria of the Bifidobacterium sp., Lactobacillus sp. or Barnesiella sp. The enhancement of beneficial bacteria may in turn lead to increased production of short chain fatty acids (SCFAs) such as acetate, propionate and butyrate, which have been shown to have many benefits in infants and young children The benefits are e.g., inhibition of pathogen bacteria, prevention of infection and diarrhea, reduced risk of allergy and metabolic disorders (see for example WO2006/130205, WO 2017/129644, WO2017/129649).

The mixtures or composition of HMOs produced according to the method described herein may be used to reduce the abundance of undesirable viruses and bacteria in the gut microbiome. Examples of pathogenic bacteria and viruses that may be reduced by the HMO mixtures described herein are including Candida albicans, Clostridium difficile, Enterococcus faecium, Escherichia coli, Helicobacter pylori, Streptococcus agalactiae, Shigella dysenteriae, Staphylococcus aureus, nora virus and rota virus. Each mixture or composition described herein can also be used to treat and/or reduce the risk of a broad range of bacterial infections of a human.

The mixtures or composition of HMOs produced according to the method described herein, may be used to increase the regeneration and viability of lyophilized probiotics, including probiotics of Bifidobacterium sp. and/or Lactobacillus sp., in particular increased regeneration and/or viability and/or shelf-life in an acidic environment, such as the stomach or acidic food products, is an advantage using the HMO mixtures described herein. Examples of Bifidobacterium sp. which may have increased regeneration and viability are Bifidobacterium animals lactis BB12 DSM 32269, Bifidobacterium animals lactis BIF6, Bifidobacterium longum DSM 32946, Bifidobacterium longum BB536, Bifidobacterium bifidum DSMZ 32403, Bifidobacterium infantis, Bifidobacterium breve DSM 33789, Bifidobacterium infantis SP37 DSM 32687, Bifidobacterium adolescentis DSM 34065 and/or Bifidobacterium animalis ssp. animalis DSM 16284 . . . . Examples of Lactobacillus sp which may have increased regeneration and viability are Lactobacillus rhamnosus GG DSM 32550, Lactobacillus rhamnosus 19070-2 DSM 26357, Lactobacillus rhamnosus GG, Lactobacillus rhamnosus LBrGG, Lactobacillus reuteri DSM 12246, Lactobacillus plantarum TIFN101, Lactobacillus gasseri Lg-36 200B FloraFit Danisco, Lactobacillus casei DSM 32382, Lactobacillus paracasei, Lactobacillus plantarum PS 128, Lactobacillus plantarum (Sacco) DSM 32383, Lactococcus lactis PAREVE, Lactobacillus paracasei ssp. Paracasei and/or Lactobacillus Probio-Tec®LGG®, LimosiLactobacillus reuteri S12 DSM 33752.

In the context of the present application “Regeneration” means the process of regaining/restoring a dried bacteria's viability (i.e., “reviving” the bacterial cells by rehydration, wherein “rehydration” means restoring fluid). This process is also sometimes referred to as “reconstitution”.

In the context of the present application “Viability” is the ability of a bacterial cell to live and function as a living cell. One way of determining the viability of bacterial cells is by spreading them on an agar plate with suitable growth medium and counting the number of colonies formed after incubation for a predefined time (plate counting). Alternatively, FACS analysis may be used.

In the context of the present application “Improving the regeneration” of Bifidobacterium sp. and/or Lactobacillus sp bacteria means to increase the amount (number) of Bifidobacterium sp. and/or Lactobacillus sp. bacteria successfully regenerating/reviving compared to the respective control (i.e., the amount/number of Bifidobacterium sp. and/or Lactobacillus sp. bacteria without the addition of HMO).

In the context of the present application “Improving the viability” of Bifidobacterium sp. and/or Lactobacillus sp. bacteria means to increase the amount (number) of viable Bifidobacterium sp. and/or Lactobacillus sp. bacteria compared to the respective control (i.e., the amount/number of Bifidobacterium sp. and/or Lactobacillus sp. bacteria without the addition of HMO).

In the context of the present application “acidic” means having a pH below 7.0 (for example, having a pH≤6.0, or ≤5.0, or ≤4.0, or ≤3.0, or in the range of 1.0-6.0, such as from 2.0 to 5.0). The pH measured in the stomach is in the range of about 1.5-3.5. The pH measured in a healthy vagina is in the range of about 3.8-5.0. The pH of fruit juices is in the range of about 2.0-4.5.

The mixtures or composition of HMOs produced according to the method described herein, may be used to extend the shelf life of probiotics, such as Bifidobacterium sp. and/or Lactobacillus sp.

An embodiment of the present invention is a composition comprising a mixture of HMOs as described herein, in particular in the section “Mixtures of HMOs”, and one or more probiotics.

Preferably, the probiotic is a Bifidobacterium sp. and/or Lactobacillus sp. such as any of the specific species mentioned above.

The mixtures or composition of HMOs produced according to the method described herein, may be used to improve the flowability of a powder or decrease the viscosity of a liquid.

The mixtures or composition of HMOs produced according to the method described herein, are used in a nutritional composition. Nutritional compositions are for example, an infant formula, a rehydration solution, or a dietary maintenance, medical nutrition or supplement for elderly individuals or immunocompromised individuals. Macronutrients such as edible fats, carbohydrates and proteins can also be included in such anti-infective compositions. Edible fats include, for example, coconut oil, soy oil and monoglycerides and diglycerides. Carbohydrates include, for example, glucose, edible lactose and hydrolysed cornstarch. Proteins include, for example, soy protein, whey, and skim milk. Vitamins and minerals (e. g. calcium, phosphorus, potassium, sodium, chloride, magnesium, manganese, iron, copper, zinc, selenium, iodine, and Vitamins A, E, D, C, and B complex) can also be included in such anti-infective compositions.

The present invention also relates to the use of a mixture or composition according to the present invention as a dietary supplement and/or medical nutrition.

In embodiments, the invention relates to the use of a mixture or composition according to the present invention in infant nutrition.

### Sequences

The current application contains a sequence listing in text format and electronical format which is hereby incorporated by reference.

An overview of the SEQ ID NOs used in the present application can be found in table 1 (α-1,3-fucosyltransferase protein sequences) and table 4 (promoter sequences), additional sequences described in the application is the colanic acid gene cluster (gmd-wcaG-wcaH-wcal-manC-manB SEQ ID NO: 12), the amino acid sequence of the fucosyltransferase FutB from Helicobacter pylori (SEQ ID NO: 6, GenBank ID WP_000487430.1) and CafD from (SEQ ID NO: 41) and FucT109 (SEQ ID NO: 42), DNA sequences encoding the α-1,3-fucosyltransferases (SEQ ID NO: 7 to 11), including FutB (SEQ ID NO: 40), CafD (SEQ ID NO: 44) and FucT109 (SEQ ID NO: 43), the DNA sequence encoding the colanic acid gene cluster from E. coli (SEQ ID NO: 12) and the β-1,3-N-acetylglucosaminyltransferase LgtA from N. meningitidis (SEQ ID NO: 13), β-1,4-galactosyltransferases galT from H. pylori (SEQ ID NO: 14).

### Items

1. A genetically engineered cell capable of producing one or more fucosylated HMOs, comprising a recombinant nucleic acid sequence encoding a fucosyltransferase with α-1,3-fucosyltransferase activity, wherein the glycosyltransferase is selected from the group consisting of
   a. Camp1, with an amino acid sequence according to SEQ ID NO: 1 or a functional homologue thereof with an amino acid sequence that is at least 80% identical to SEQ ID NO: 1;
   b. Parm1 with an amino acid sequence according to SEQ ID NO: 2 or a functional homologue thereof with an amino acid sequence that is at least 80% identical to SEQ ID NO: 2;
   c. Hpull1 with an amino acid sequence according to SEQ ID NO: 3, or a functional homologue thereof with an amino acid sequence that is at least 80% identical to SEQ ID NO: 3;
   d. Med1, with an amino acid sequence according to SEQ ID NO: 4 or a functional homologue thereof with an amino acid sequence that is at least 80% identical to SEQ ID NO: 4; and
   e. Hacin2 with an amino acid sequence according to SEQ ID NO: 5 or a functional homologue thereof with an amino acid sequence that is at least 80% identical to SEQ ID NO: 5.
2. The genetically engineered cell according to item 1, wherein the fucosyltransferase primarily fucosylates the N-acetylglucoseamine (GlcNAc) moiety of an acceptor oligosaccharide in an α-1,3 linkage, such as the N-acetylglucoseamine (GlcNAc) moiety LNnT.
3. The genetically engineered cell according to item 1 or 2, wherein the fucosyltransferase has low or no activity on the glucose moiety in the reducing end of an acceptor molecule, such as lactose or an HMO, such as LNT-II or LNnT, in particular LNnT.
4. The genetically engineered cell according to any one of items 1 to 3, wherein the one or more fucosylated HMOs is LNFP-III.
5. The genetically engineered cell according to any of the preceding items, wherein at least 30% of the molar content of the total HMOs produced by said cell is LNFP-III.
6. The genetically engineered cell according to any of the preceding items, wherein less than 5%, preferably less than 3% of the molar content of the total HMOs produced by the cell is LNFP-VI and/or LNDFH-III.
7. The genetically engineered cell according to any of the preceding items, wherein the wherein the HMOs produced by said cell is essentially free of LNFP-VI.
8. The genetically engineered cell according to any of the preceding items, wherein the genetically engineered cell comprises one or more further recombinant nucleic acids encoding one or more heterologous glycosyltransferases.
9. The genetically engineered cell according to item 8, wherein the genetically engineered cell comprises a recombinant nucleic acid sequence encoding a β-1,4-galactosyltransferase.
10. The genetically engineered cell according to item 8 or 9, wherein the genetically engineered cell comprises a recombinant nucleic acid sequence encoding a β-1,3-N-acetyl-glucosaminyltransferase.
11. The genetically engineered cell according to any one of items 9 or 10, wherein the β-1,3-N-acetylglucosaminyltransferase is from Neisseria meningitidis and the β-1,4-galactosyltransferase is from Helicobacter pylori.
12. The genetically engineered cell according to any one of items 9 to 11, wherein the 1,3-N-acetylglucosaminyltransferase has an amino acid sequence according to SEQ ID NO: 13, or a functional homologue thereof with an amino acid sequence that is at least 80% identical to SEQ ID NO: 13 and the β-1,4-galactosyltransferase is has an amino acid sequence according to SEQ ID NO: 14, or a functional homologue thereof with an amino acid sequence that is at least 80% identical to SEQ ID NO: 14.
13. The genetically engineered cell according to any of the preceding items, wherein recombinant nucleic acid sequences individually are under the control of a promoter selected from the group consisting of PglpF, Plac, PmglB_70UTR, PglpA_70UTR and PglpT_70UTR (SEQ ID NOs: 28, 37, 25, 26 and 27, respectively) and variants thereof.
14. The genetically engineered cell according to item 13, wherein the promoter is a strong promoter selected from the group consisting of SEQ ID NOs 16, 17, 18, 19, 20, 21, 22, 23, 24, 25, 26 and 27.
15. The genetically engineered cell according to any of the preceding items, wherein the cell comprises one, two, three or more genomic copies of the recombinant nucleic acid sequence encoding the glycosyltransferase selected from the group consisting of Camp1, Parm1, Hpull1, Med1, and Hacin2 with an amino acid sequence according to SEQ ID NO: 1, 2, 3 4 or 5, or a functional homologue thereof with an amino acid sequence that is at least 80% identical to SEQ ID NO: 1, 2, 3, 4 or 5.
16. The genetically engineered cell according to any of the preceding items, wherein the recombinant nucleic acid sequence encoding the glycosyltransferase selected from the group consisting of Camp1, Parm1, Hpull1, Med1, and Hacin2 with an amino acid sequence according to SEQ ID NO: 1, 2, 3, 4 or 5, or a functional homologue thereof with an amino acid sequence that is at least 80% identical to SEQ ID NO: 1, 2, 3, 4 or 5, is encoded on a plasmid.
17. The genetically engineered cell according to item 16, wherein the plasmid is high copy number plasmid.
18. The genetically engineered cell according to item 16 or 17, wherein the plasmid is a pUC57 plasmid.
19. The genetically engineered cell according to any of the preceding items, wherein the cell comprises one, two, three or more genomic copies and a plasmid borne copy of the recombinant nucleic acid sequence encoding the glycosyltransferase selected from the group consisting of Camp1, Parm1, Hpull1, Med1, and Hacin2 with an amino acid sequence according to SEQ ID NO: 1, 2, 3, 4 or 5, or a functional homologue thereof with an amino acid sequence that is at least 80% identical to SEQ ID NO: 1, 2, 3, 4 or 5.
20. The genetically engineered cell according to any of the preceding items, wherein the cell overexpresses at least one enzyme in the de novo GDP-fucose pathway responsible for the formation of GDP-fucose.
21. The genetically engineered cell according to item 20, wherein the one or more enzyme is selected from the group consisting of mannose-6 phosphate isomerase (manA), phosphomannomutase (manB), mannose-1-phosphate guanylyltransferase guanylyltransferase (manC), GDP-mannose-4,6-dehydratase (gmd) and GDP-L-fucose synthase (wcaG).
22. The genetically engineered cell according to any of the preceding items, wherein the cell further comprises a recombinant nucleic acid sequence according to SEQ ID NO: 12 encoding the colanic acid (CA) gene cluster.
23. The genetically engineered cell according to any one of the preceding items, wherein the cell further comprises a nucleic acid sequence encoding an MFS transporter protein capable of exporting the one or more fucosylated HMOs into the extracellular medium, in particular LNFP-III.
24. The genetically modified cell according to item 23, wherein the MFS transporter protein is the yjhB, YhhS or YebQ protein or a functional variant thereof, such as a protein with at least 80% identity to the protein with UniProt accession nr P39352 or P37621 or P76269.
25. The genetically engineered cell according to any of the preceding items, wherein said engineered cell is a microorganism.
26. The genetically engineered cell according to any of the preceding items, wherein said engineered cell is a bacterium or a fungus.
27. The genetically engineered cell according to item 26, wherein said fungus is selected from a yeast cell of the genera Komagataella, Kluyveromyces, Yarrowia, Pichia, Saccaromyces, Schizosaccharomyces or Hansenula or from a filamentous fungus of the genera Aspargillus, Fusarium or Thricoderma.
28. The genetically engineered cell according to item 26, wherein said bacterium is selected from the group consisting of Escherichia sp., Bacillus sp., Lactobacillus sp., Corynebacterium sp. and Campylobacter sp.
29. The genetically engineered according to any of items 25 to 28, wherein said engineered cell is selected from the group consisting of Escherichia Coli, Bacillus subtilis, Lactobacillus lactis, Corynebacterium glutamicum, Yarrowia lipolytica, Pichia pastoris, and Saccharomyces cerevisiae.
30. The genetically engineered cell according to item 29, wherein said engineered cell is a microorganism is E. coli.
31. The genetically engineered cell according to any of the preceding items, wherein the glycosyltransferase is selected from Camp1, Parm1, Hpull1 and Med1 with an amino acid sequence according to SEQ ID NO: 1, 2, 3 or 4, or a functional homologue thereof with an amino acid sequence that is at least 80% identical to SEQ ID NO: 1, 2, 3 or 4 [Camp1, Pam1 Hpull1 and Med1 amino acid], and wherein at least 75% of the molar content of the total HMOs produced by said cell is LNFP-III.
32. The genetically engineered cell according to any of the preceding items, the glycosyltransferase is selected from Camp1, Hpull1 and Med1 with an amino acid sequence according to SEQ ID NO: 1, 3 or 4, or a functional homologue thereof with an amino acid sequence that is at least 80% identical to SEQ ID NO: 1, 3 or 4 [Camp1, Hpull1 and Med1 amino acid], and wherein the total HMOs produced by said cell is essentially free of LNFP-VI.
33. The genetically engineered cell according to any of the preceding items, the glycosyltransferase is selected from Camp1, Hpull1 and Med1 with an amino acid sequence according to SEQ ID NO: 1, 3 or 4, or a functional homologue thereof with an amino acid sequence that is at least 80% identical to SEQ ID NO: 1, 3 or 4 [Camp1, Hpull1 and Med1 amino acid], and wherein the total HMOs produced by said cell is essentially free of LNDFH-III.
34. The genetically engineered cell according to any of the preceding items, wherein the cell produces a mixture of HMOs comprising LNFP-III and LNnT.
35. The genetically engineered cell according to any of the preceding items, wherein the cell produces a mixture of HMOs consisting essentially of 30-99% LNFP-III, 0-65% LNnT, 0-20% 3-FL and 0-10% pLNnH in total adding up to 100% molar content.
36. The genetically engineered cell according to any of the preceding items, wherein the cell produces at least 50% LNFP-III of the molar content of the total HMOs produced by said cell and the remaining 50% of the total HMO produced by said cell is by-product HMOs selected from the group consisting of LNDFH-III, 3FL, LNnT and pLNnH.
37. A method for producing one or more fucosylated HMOs, said method comprising culturing a genetically engineered cell according to any of items 1 to 35.
38. The method according to any one of items 37, wherein the method comprises cultivating the genetically engineered cell in the presence of an energy source selected from the group consisting of glucose, sucrose, fructose, xylose and glycerol.
39. The method according to any one of items 37 or 38, wherein lactose is added during the cultivation of the genetically engineered cells as a substrate for the HMO formation.
40. The method according to item 37 to 39, wherein the fucosylated HMO is LNFP-III.
41. The method according to item 37 to 40, wherein the method produces a mixture of HMOs and wherein said mixture is optionally purified to at least 85% purity.
42. The method according to item 37 to 40, wherein the LNFP-III produced by the method is purified to at least 90% purity.
43. Use of an enzyme with α-1,3-fucosyltransferase activity in production of a fucosylated product, wherein the enzyme is selected from the group consisting of Camp1, Parm1, Hpull1, Med1, and Hacin2 with an amino acid sequence according to SEQ ID NO: 1, 2, 3, 4 or 5, or a functional homologue thereof with an amino acid sequence that is at least 80% identical to SEQ ID NO: 1, 2, 3, 4 or 5.
44. The use of an enzyme according to item 43, wherein the fucosylated product is one or more fucosylated oligosaccharides.
45. The use of an enzyme according to any of items 43 or 44, wherein the fucosylated product is one or more HMOs.
46. The use of an enzyme according to item 45, wherein the fucosylated product is LNFP-III.
47. The use of an enzyme according to item 46, wherein LNFP-III is essentially the only fucosylated HMO produced when LNnT is used as acceptor substrate for the α-1,3-fucosyltransferase.
48. The use of an enzyme according to item 43, wherein the fucosylated product is one or more fucosylated polypeptides.
49. A mixture of HMOs consisting essentially of LNFP-III and LNnT and less than 20% of 3FL, LNDFH-III and/or pLNnH.
50. The mixture of HMOs according to item 49, consisting essentially of 30-99% LNFP-III, 0-65% LNnT, 0-20% 3-FL, 0-10% LNDFH-III and 0-10% pLNnH in total adding up to 100% molar content.
51. The mixture of HMOs according to item 49, consisting essentially of 75-99% LNFP-III and 1-25% LNnT, in total adding up to 100% molar content.
52. The mixture of HMOs according to item 49, consisting essentially of 55-85% LNFP-III and 15-45% LNnT, in total adding up to 100% molar content.
53. The mixture of HMOs according to item 49, consisting essentially of 65-85% LNFP-III and 15-35% LNnT, in total adding up to 100% molar content.
54. The mixture of HMOs according to item 49, consisting essentially of 25-60% LNFP-III, 35-65% LNnT and 2-8% pLNnH, in total adding up to 100% molar content.
55. The mixture of HMOs according to item 49, consisting essentially of 85-95% LNFP-III, 0-5% LNnT and 2-15% LNDFH-III, in total adding up to 100% molar content.
56. The mixture of HMOs according to item 49, consisting essentially of 75-85% LNFP-III, and 15-25% 3FL in total adding up to 100% molar content.
57. The mixture of HMOs according to any one of items 49 to 55, wherein the mixture is essentially free of LNFP-VI.
58. A composition comprising a mixture of HMOs according to items 49 to 57.
59. The composition according to item 58, wherein the mixture further comprises a one or more probiotics.
60. The composition according to item 59, wherein the probiotic is a Bifidobacterium sp and/or a Lactobacillus sp.
61. The composition according to item 59 or 60, wherein the probiotic is selected from the group consisting of Bifidobacterium animals lactis BB12 DSM 32269, Bifidobacterium animals lactis BIF6, Bifidobacterium longum DSM 32946, Bifidobacterium longum BB536, Bifidobacterium bifidum DSMZ 32403, Bifidobacterium infantis, Bifidobacterium breve DSM 33789, Bifidobacterium infantis SP37 DSM 32687, Bifidobacterium adolescentis DSM 34065, Bifidobacterium animalis ssp. animalis DSM 16284, Lactobacillus rhamnosus GG DSM 32550, Lactobacillus rhamnosus 19070-2 DSM 26357, Lactobacillus rhamnosus GG, Lactobacillus rhamnosus LBrGG, Lactobacillus reuteri DSM 12246, Lactobacillus plantarum TIFN101, Lactobacillus gasseri Lg-36 200B FloraFit Danisco, Lactobacillus casei DSMZ 32382, Lactobacillus paracasei, Lactobacillus plantarum PS 128, Lactobacillus plantarum (Sacco) DSM 32383, Lactococcus lactis PAREVE, Lactobacillus paracasei ssp. Paracasei and Lactobacillus Probio-Tec®LGG®, LimosiLactobacillus reuteri S12 DSM 33752.
62. The composition according to item 59 or 60. Wherein the probiotic is Lactobacillus casei DSMZ 32382.
63. Use of a mixture or composition according to any of items 49 to 62 as a dietary supplement and/or medical nutrition.
64. Use of a mixture or composition according to any of items 49 to 62 in infant nutrition.

### EXAMPLES

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY)

The embodiments described below are selected to illustrate the invention and are not limiting the invention in any way.

### Enzymes:

Screening of 50 enzymes with fucosyltransferase activity provided 6 enzymes with α-1,3-fucosyltransferase activity, which were also capable of producing the complex HMO LNFP-III. The GenBank ID and origin of the six fucosyltransferases are provided in table 5.

TABLE 5
List of the enzymes tested in the
framework of the present invention

| Enzyme | | SEQ | |
|---|---|---|---|
| Name | GenBank ID* | ID NO: | Origin |
| Camp1 | MBP3676691.1 | 1 | Campylobacter sp. |
| Parm1 | WP_129943149.1 | 2 | Parabacteroides merdae |
| Hpull1 | WP_065831246.1 | 3 | Helicobacter pullorum |
| Med1 | WP_087337236.1 | 4 | Mediterranea sp. An20 |
| Hacin2 | WP_104687422.1 | 5 | Helicobacter acinonychis |
| FutB** | WP_000487430.1 | 6 | Helicobacter pylori |
| CafD*** | AAP76669.1 | 41 | Helicobacter hepaticus |
| | | | ATCC 51449 |
| FucT109\hat{ } | CAH09151.1 | 42 | Bacteroides fragilis |
| | | | ATCC 25285 |

*the sequences used in the present application may be truncated at the N- or C-terminal as compared to the GenBank sequence these are represented by the SEQ ID NO.
**FutB is known to produce a mixture of LNFP-III and LNFP-VI (Dumon et al 2004 Biotechnol. Prog. 20: 412-419).
***CafD has been suggested to produce LNFP-III in WO2016/040531
\hat{ }FucT 109 has been suggested to produce LNFP-III in WO 2019/008133

### Strains

The strains (genetically engineered cells) constructed in the present application were based on Escherichia coli K-12 DH1 with the genotype: F⁻, A⁻, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, IacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene. Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186:2673-81 and Warming et al 2005 Nucleic Acids Res. 33 (4): e36) with specific selection marker genes and screening methods.

To obtain an LNnT producing strain the MDO strain was further engineered by chromosomally integrating a beta-1,3-GlcNAc transferase (LgtA from Neisseria meningitidis, homologous to NCBI Accession nr. WP_033911473.1 and shown as SEQ ID NO: 13) and a beta-1,4-galactosyltransferase (GalT from Helicobacter pylori, homologous to GenBank ID WP_001262061.1 and shown as SEQ ID NO: 14) both under the control of a PglpF promoter (SEQ ID NO: 28), this strain is named the LNnT strain.

Codon optimized DNA sequences encoding individual α-1,3-fucosyltransferases were genomically integrated into the LNnT strain.

The genotypes of the background strain (MDO), the LNnT strain and the α-1,3-fucosyltransferase expressing strains capable of producing LNFP-III are provided in Table 6.

TABLE 6
Genotypes of the strains, capable of producing
LNFP-III, used in the present examples.

| | | α-1,3FT* cDNA |
|---|---|---|
| Strain ref | Genotype | SEQ ID NO |
| MDO | F−λ− ΔendA1 ΔrecA1 ΔrelA1 ΔgyrA96 Δthi-1 glnV44 | — |
| | hsdR17(rK−mK−) ΔlacZ wcaF::Plac ΔnanKETA ΔlacA | |
| | ΔmelA ΔwcaJ ΔmdoH | |
| LNnT | MDO, 2x lgtA-PglpF¹, 1x galT-PglpF² | — |
| Camp1_1 | LNnT, Camp1-PglpF, CA-PglpF³ | 7 |
| Camp1_2 | LNnT, Camp1-PglpF, 1x pUC57⁴-Camp1-PglpF-amp, | 7 |
| | CA-PglpF³ | |
| Parm1_1 | LNnT, Parm1-PglpF, CA-PglpF³ | 8 |
| Parm1_2 | LNnT, 2x Parm1-PglpF, CA-PglpF³ | 8 |
| Parm1_3 | LNnT, 2x Parm1-PglpF, 1x pUC57⁴-Parm1-PglpF-amp, | 8 |
| | CA-PglpF³ | |
| Hpull1 | LNnT, Hpull1-PglpF, CA-PglpF³ | 9 |
| Med1_1 | LNnT, Med1-PglpF, CA-PglpF³ | 10 |
| Med1_2 | LNnT, 2x Med1-PglpF, CA-PglpF³ | 10 |
| Med1_2 | LNnT, 2x Med1-PglpF, 1x pUC57⁴-Med1-PglpF-amp | 10 |
| | CA-PglpF³ | |
| FutB** | LNnT, FutB-PglpF, CA-PglpF³ | 40 |
| CafD*** | LNnT, CafD-PglpF, CA-PglpF³ | 44 |
| FucT109 | LNnT, FucT109-PglpF, CA-PglpF³ | 43 |

*1,3FT is an abbreviation of α-1,3-fucosyltransferase, and the sequence is inserted into the genome of the host strain.
¹lgtA-PglpF - two genomically inserted copies of a gene encoding β-1,3-N-acetyl-glucosaminyltransferase (SEQ ID NO: 13) under control of a PglpF promoter.
²galT-PglpF - one genomically inserted gene encoding β-1,4-Galactosyltransferase (SEQ ID NO: 14) under control of a PglpF promoter.
³CA = extra colanic acid gene cluster (gmd-wcaG-wcaH-wcal-manC-manB, SEQ ID NO: 12) under the control of a PglpF promoter at a locus that is different than the native locus.
⁴pUC57, is high-copy number (>300) plasmid having the ColE1/pMB1/pBR322/pUC origin of replication. The antibiotic resistance marker on the pUC57 vector is ampicillin. The indicated a1,3FT is expressed from the plasmid.
**FutB is known to produce a mixture of LNFP-III and LNFP-VI (Dumon et al 2004 Biotechnol. Prog. 20: 412-419).
***CafD has been suggested to produce LNFP-III in WO2016/040531

### Deep Well Assay

Deep Well Assays in the current examples were performed as originally described to Lv et al (Bioprocess Biosyst Eng 20 (2016) 39:1737-1747) and optimized for the purposes of the current invention. More specifically, the strains disclosed in the present example were screened in 96 deep well plates using a 4-day protocol. During the first 24 hours, precultures were grown to high densities (OD600 up to 5) and subsequently transferred to a medium that allowed induction of gene expression and product formation.

More specifically, during day 1, fresh precultures were prepared using a basal minimal medium (BMM) (pH 7.0) supplemented with magnesium sulphate (0.12 g/L), thiamine (0.004 g/L) and glucose (5.5 g/L). Basal Minimal medium had the following composition: NaOH (1 g/L), KOH (2.5 g/L), KHzPO4 (7 g/L), NH&HzPO4 (7 g/L), Citric acid (0.5 g/l), trace mineral solution (5 mL/L). The trace mineral stock solution contained; ZnSO~*7H~O 0.82 g/L, Citric acid 20 g/L, Mn$04*H&O 0.98 g/L, FeSO4*7H&O 3.925 g/L, CuSO4*5H~O 0.2 g/L. The pH of the Basal Minimal Medium was adjusted to 7.0 with 5 N NaOH and autoclaved. The precultures were incubated for 24 hours at 34° C. and 1000 rpm shaking and then further transferred to 0.75 ml of a new BMM (pH 7,5) to start the main culture. The new BMM was supplemented with magnesium sulphate (0.12 g/L), thiamine (0.02 g/L), a bolus of glucose solution (0.1-0.15 g/L) and a bolus of lactose solution (5-20 g/L) Moreover, a 20% stock solution of sucrose (40-45 g/L) or maltodextrin (19-20 g/L) was provided as carbon source, accompanied by the addition of a specific hydrolytic enzyme, sucrose hydrolase or glycoamylase, respectively, so that glucose was released at a rate suitable for carbon-limited growth and similar to that of a typical fed-batch fermentation process. The main cultures were incubated for 72 hours at 28° C. and 1000 rpm shaking. For the analysis of total broth, the 96 well plates were boiled at 100° C., subsequently centrifuged, and finally the supernatants were analysed by HPLC.

### Fermentation-Amber

The E. coli strains were cultivated in 250 mL fermenters (Ambr250 HT Bioreactor system, Sartorius) starting with 100 ml of mineral culture medium consisting of 30 g/L glucose and a mineral medium comprised of NH₄H₂PO₄, KH₂PO₄, MgSO₄×7H₂O, KOH, NaOH, citric acid, trace element solution, antifoam and thiamine. The dissolved oxygen level was kept at 20% by a cascade of first agitation and then airflow starting at 700 rpm (up to max 4500 rpm) and 1 WM (up to max 3 VVM). The pH was kept at 6.8 by titration with 8.5% NH₄OH solution. The cultivations were started with 2% (v/v) inoculums from pre-cultures comprised of 10 g/L glucose, (NH4)2HPO4, KH2PO4, MgSO4×7H2O, KOH, NaOH, citric acid, trace element solution, antifoam and thiamine. After depletion of the glucose contained in the basal minimal medium, a feed solution containing glucose, MgSO₄×7H₂O, H₃PO₄ and trace mineral solution was continuously added to the fermenter at a rate that maintained carbon-limiting conditions. The temperature was initially at 33° C. but was dropped to 30° C. with a 3-hour linear ramp initiated 12 hours after the start of the feed. Lactose was added as bolus additions of 25% lactose monohydrate solution 6 hours after feed start and then every 19 hours to keep lactose from becoming a rate limiting factor. The growth, metabolic activity and metabolic state of the cells was followed by on-line measurements of agitation, dissolved oxygen tension, reflectance, NH₄OH base addition, O₂ uptake rate and CO₂ evolution rate. Throughout the fermentations, samples were taken to determine the concentration of HMO products, lactose and other minor by-products using HPLC.

### Fermentation-Dasbox

The E. coli strains were cultivated in 100 mL fermenters (Dasbox, Eppendorf) starting with 100 mL of mineral culture medium consisting of 30 g/L glucose and a mineral medium comprised of NH₄H₂PO₄, KH₂PO₄, MgSO₄×7H₂O, KOH, NaOH, citric acid, trace element solution, antifoam and thiamine. The dissolved oxygen level was kept at 20% by a cascade of first agitation and then airflow starting at 700 rpm (up to max 2000 rpm) and 1 VVM (up to max 3 WVM). The pH was kept at 6.8 by titration with 8.5% NH₄OH solution. The cultivations were started with 2% (v/v) inoculums from pre-cultures comprised of 10 g/L glucose, (NH₄)₂HPO4, KH₂PO₄, MgSO₄×7H₂O, KOH, NaOH, citric acid, trace element solution, antifoam and thiamine. After depletion of the glucose contained in the basal minimal medium, a feed solution containing glucose, MgSO₄×7H₂O, H₃PO₄ and trace mineral solution was continuously added to the fermenter at a rate that maintained carbon-limiting conditions. The temperature was initially at 33° C. but was dropped to 25° C. initiated 15 min after the start of the feed. Lactose was added as bolus additions of 25% lactose monohydrate solution at feed start and also together with the glucose feed to keep lactose from becoming a rate limiting factor. The growth, metabolic activity and metabolic state of the cells was followed by on-line measurements of agitation, dissolved oxygen tension, reflectance, NH₄OH base addition, O₂ uptake rate and CO₂ evolution rate. Throughout the fermentations, samples were taken to determine the concentration of HMO products, lactose and other minor by-products using HPLC.

### Example 1-In Vivo LNFP-III Synthesis

Genetically modified cells expressing individual α-1,3-fucosyltransferase enzymes were screened for their ability to produce the fucosylated HMO LNFP-III.

Five enzymes (table 7) were compiled for testing their ability to synthesize LNFP-III when introduced into a genetically modified cells that produce LNnT and GDP-Fucose.

Genetically modified strains expressing the six individual α-1,3-fucosyltransferases (table 5) were generated as described in the “Method” section. The cells were screened in the deep well assay setup as described in the “Method” section.

Table 6 lists the genotype of the strains capable of producing LNFP-III. The molar content of individual HMOs produced by the strains was measured by HPLC.

The results of the LNFP-III producing cells are shown in table 7 as the fraction of the total molar HMO content (in percentage, %) produced by each strain.

TABLE 7
Content of individual HMO's as % of total HMO molar (mM) content produced by each strain.

| | FT | | | | | | |
|---|---|---|---|---|---|---|---|
| Enzyme | Copy nr | LNFP-III | LNDFH-III | 3-FL | LNnT | LNFP-VI | pLNnH |
| CafD | 1 | 0.0 ± 0.0 | 0.0 ± 0.0 | 0.0 ± 0.0 | 81.9 ± 1.3 | 0.0 ± 0.0 | 18.1 ± 1.3 |
| FutT109 | 1 | 26.6 ± 3.5 | 23.9 ± 1.8 | 2.0 ± 1.8 | 28.7 ± 1.6 | 18.0 ± 2.6 | 0.8 ± 0.7 |
| FutB | 1 | 6.2 ± 0.5 | 0.0 ± 0.0 | 1.3 ± 0.1 | 78.9 ± 0.4 | 0.5 ± 0.5 | 13.2 ± 0.2 |
| Hacin2_1 | 1 | 31.3 ± 5.5 | 0.0 ± 0.0 | 0.3 ± 0.6 | 63.4 ± 4.2 | 0.0 ± 0.0 | 4.9 ± 1.6 |
| Med1_1 | 1 | 52.0 ± 3.4 | 0.0 ± 0.0 | 0.5 ± 0.7 | 41.5 ± 2.6 | 0.0 ± 0.0 | 5.9 ± 1.2 |
| Parm1_1 | 1 | 76.1 ± 4.0 | 2.0 ± 1.9 | 0.5 ± 0.7 | 21.2 ± 4.4 | 0.0 ± 0.0 | 0.1 ± 0.2 |
| Hpull1_1 | 1 | 76.8 ± 0.4 | 0.0 ± 0.0 | 0.0 ± 0.0 | 23.2 ± 0.4 | 0.0 ± 0.0 | 0.0 ± 0.0 |
| Camp1_1 | 1 | 97.6 ± 3.4 | 0.2 ± 0.6 | 0.4 ± 0.6 | 1.8 ± 2.9 | 0.0 ± 0.0 | 0.1 ± 0.1 |
| Camp1_2 | >50 | 96.2 ± 4.3 | 0.0 ± 0.0 | 3.8 ± 4.3 | 0.0 ± 0.0 | 0.0 ± 0.0 | 0.0 ± 0.0 |
| Parm1_2 | 2 | 81.5 ± 0.3 | 4.7 ± 0.3 | 0.0 ± 0.0 | 13.9 ± 0.4 | 0.0 ± 0.0 | 0.0 ± 0.0 |
| Parm1_3 | >50 | 85.7 ± 3.1 | 8.9 ± 3.3 | 0.5 ± 1.2 | 5.0 ± 5.6 | 0.0 ± 0.0 | 0.0 ± 0.0 |
| Med1_2 | 2 | 89.9 ± 0.4 | 0.0 ± 0.0 | 0.0 ± 0.0 | 10.1 ± 0.4 | 0.0 ± 0.0 | 0.0 ± 0.0 |
| Med1_3 | >50 | 81.8 ± 0.8 | 0.0 ± 0.0 | 18.2 ± 0.8 | 0.0 ± 0.0 | 0.0 ± 0.0 | 0.0 ± 0.0 |

No additional HMOs beyond the ones indicated in table 7 was identified in the deep well assay.

From the data presented in table 7 it can be seen that the five novel enzymes Camp1, Parm1, Hpull1, Med1 and Hacin2 can transfer a fucosyl unit onto the GlcNAc moiety of LNnT in an α-1,3 linkage to form LNFP-III at a level above 30% of the total HMO as compared to the prior art enzyme FutB and FucT109 which only produce 6% and 26% LNFP-III of the total HMO, respectively. CafD which was purported to produce LNFP-III in WO2016/040531 appears to be unable to fucosylate LNnT or lactose in the present assay, as it does not produce any fucosylated HMOs.

None of the five novel enzymes produce any LNFP-VI at all, whereas FucT109 produce 18% LNFP-VI clearly indicating the FucT109 has similar specificity to both the GlcNAc and Glc moiety of LNnT. Likewise, FutB was found to produce minor amounts of LNFP-VI as well as more 3-FL with a single copy of the enzyme compared to the 5 novel enzymes, indicating the FutB also have some activity to the terminal glucose (Glc) moiety of LNnT.

Furthermore, the enzymes Camp1, Hpull1, Med1 and Hacin2 also do not produce any LNDFH-Ill indicating that these enzymes are highly specific for the GlcNAc moiety in LNnT. In contrast FutT109 produce 24% LNDFH-III.

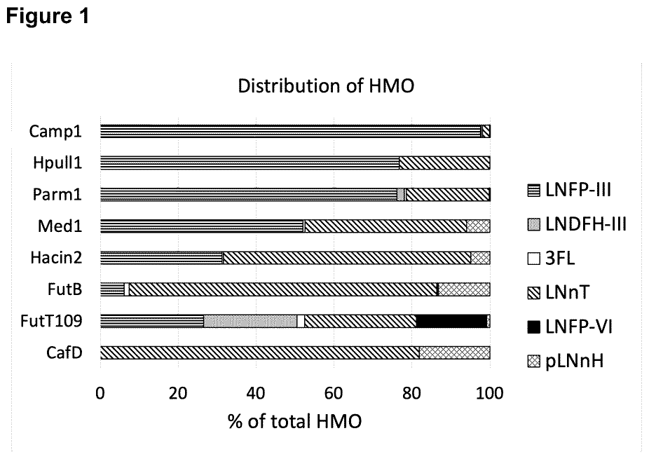

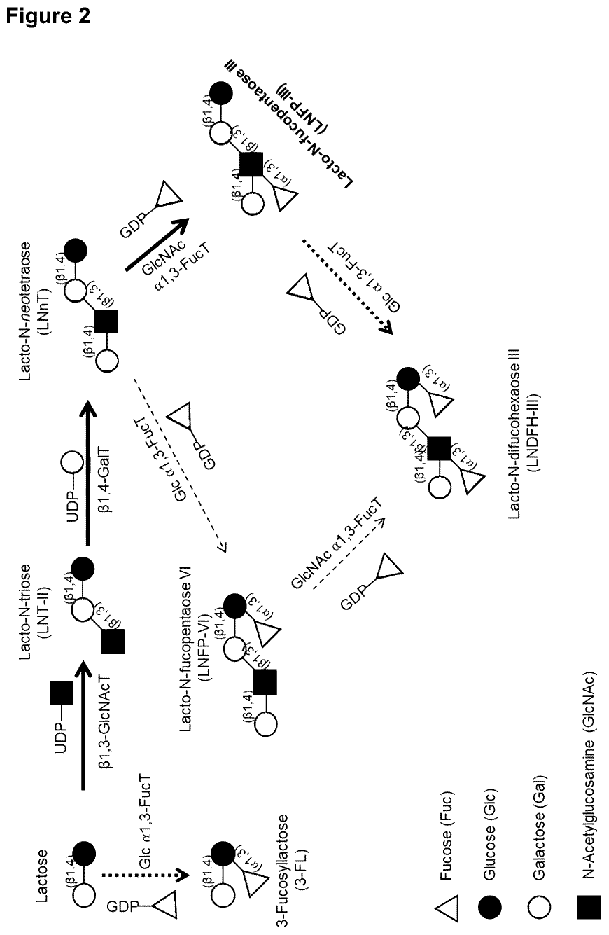

FIG. 1 illustrates the data from the single copy strains of table 7, clearly illustrating the difference in LNFP-III production from the strains with the five novel enzymes compared to the prior art enzymes FutB, CafD and FucT109.

From table 7 is can be seen that increasing the copy number of Camp1 to a high copy number expressed from a plasmid (Camp1_2) results in a slight production of 3-FL, while abolishing production of LNnT from the strain indicating that the formation LNnT in this strain is a limiting factor when the Camp1 copy nr is high.

Increasing the copy number of Parm1 to two genomic copies and further to also include a high copy plasmid (pUC57-Parm1-PglpF), increased the relative amount of LNFP-III produced from 76% to 81% to 86% of the total HMO content, combined with a decrease in the amount of LNnT produced from 21% to 14% to 5%, respectively. The increase in copy nr of Parm1 also increased the formation of LNDFH-III indicating that increased activity also increased the fucosylation of the Glc moiety in LNnT.

Increasing the copy number of Med1 to two genetic copies (strain Med1_2) increased the LNFP-III production from 52% to 90%. With introduction of a high copy plasmid (pUC57-Med1-PglpF-amp) encoding Med1 (strain Med1_3) a slight decrease in LNFP-III level down to 82% was observed and 18% 3-FL was produced instead. Given a high copy number for the Med1 gene (strain Med1_8), all the LNnT produced by the cell was fucosylated, hence the formation of LNnT by the cell became the rate limiting step in the production of LNFP-III, potentially causing Med1 to fucosylate lactose instead of LNnT, thereby further decreasing the LNnT formation by consuming the lactose, leading to the decrease in LNFP-III.

Accordingly, the copy number variation may be used to tailor specific HMOs mixtures, in this case a mixture comprising of LNFP-III, LNnT and/or 3-FL in different ratios, depending on the need for the specific product. Increasing the copy number of Camp1, Hpull1, Hacin2 and Med1 did not result in production of any additional fucosylated HMO species with an LNnT backbone neither did any of the strains with increased copy number produce 3-FL except for Med1 with the high copy number plasmid, which was most likely due to the formation of LNnT becoming the rate limiting step in the production process.

Absence of alternative fucosylated species in the produced mixture is highly advantageous and preferred if it is desired to purify the produced LNFP-III. Also, the low levels of LNnT achieved by expressing the Camp1 and Med1 genes (high copy number) is beneficial if it is desired to obtain pure LNFP-III. If med! Is applied in high copy nr. the data indicate the enzymes forming LNnT should potentially be increased together with the Med1 enzyme to secure sufficient formation of LNnT.

### Example 2-Fermentation Using Camp1_1 α-1,3-Fucosyltransferase Strain for LNFP-III Production

To confirm the HMO profile observed in the deep well assays, and especially the content of LNFP-III in the total HMO produced, the Camp1_1 strain of example 1, containing a single genomic copy of Camp1, was fermented using the ambr fermentation as described in the “Method” section above. The results are shown in table 8.

TABLE 8
Content of individual HMO's as % of
total HMO content produced by the strain

| Strain | | LNT- | LNFP- | LNFP- | LNDFH- | |
|---|---|---|---|---|---|---|
| ref. | 3-FL | II | III | VI | III | LNnT |
| Camp1_1 | 0 | 0 | 98 | 0 | 0 | 2 |

From the data presented table 8, it can be seen that the fraction of LNFP-III for the Camp1_1 strain was the same in the fermentation, as the results presented in example 1, further show the ability and suitability of Camp1 for the production of highly pure LNFP-III in fermentation.

### Example 3-Fermentation Using Med1_1 α-1,3-Fucosyltransferase Strain for LNFP-III Production

To confirm the HMO profile observed in the deep well assays, and especially the content of LNFP-III in the total HMO produced, the Med1_1 strain of example 1, containing a single genomic copy of Med1, was fermented using the dashbox fermentation as described in the “Method” section above. The results are shown in table 9. The Camp1_1 strain was run in parallel

TABLE 9
Content of individual HMO's as % of
total HMO content produced by the strain

| Strain | 3- | LNT- | LNFP- | LNFP- | LNDFH- | | |
|---|---|---|---|---|---|---|---|
| ref. | FL | II | III | VI | IIII | LNnT | pLNnH |
| Med1_1 | 0 | 3 | 24 | 0 | 0 | 61 | 12 |
| Camp1_1 | 0 | 2 | 95 | 0 | 0 | 2 | 0 |

From the data presented table 9, it can be seen that using the using the Med1_1 strain in a fermentation setup the only complex fucosylated HMO produced is LNFP-III. The conversion from LNnT to LNFP-III was somewhat lower compared to the deep well assays presented in example 2, something which may be optimized, e.g., by using a strain with multiple Med1 copies on the genome.

Camp1_1 performs almost identical to the fermentation conducted in Example 2, showing high stability in different fermentation settings.

### Example 4-Regeneration and Viability of Lyophilized Lactobacillus Species

Probiotics may be consumed as live bacteria or as a dried (e.g. lyophilized) product. Independent of the drying method, rehydration involves an important step in the recovery of dehydrated bacteria; an inadequate rehydration/regeneration step may lead to poor cell viability and a low final survival rate. Rehydration is therefore a highly critical step in the revitalization of a lyophilized culture. For both live and rehydrated bacteria, the survival of the bacteria under acidic conditions is critical since they need to pass through the acidic environment of the stomach and may also be faced with storage (shelf-life) in acidic food products.

In the present example it was tested whether the mixture of HMO produced by the LNFP-III producing strains in example 1 can provide a benefit in the rehydration (regeneration) and viability of the probiotics. The test was performed under acidic conditions to resemble the conditions bacteria have to survive when passing through the stomach or when dosed in an acidic beverage.

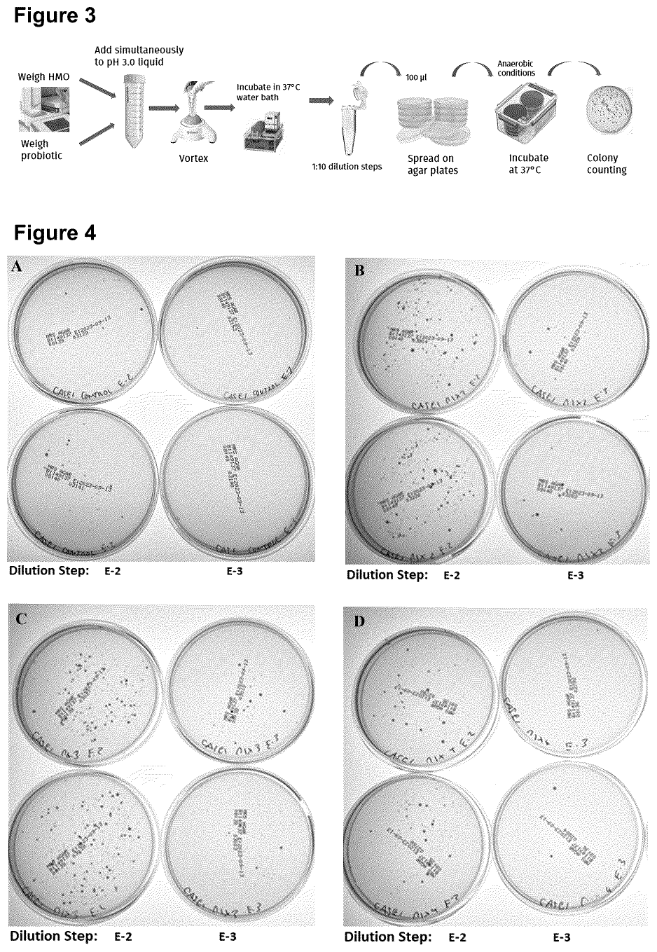

The lyophilized probiotics Lactobacillus casei (DSM 32382) (0.4 mg/ml) alone (control) or in combination with HMOs (5% w/v) as indicated in table 10, were dissolved in sterile phosphate-buffered saline (PBS, pH=3), warmed to 37° C. and vigorously mixed for about 30 sec until no visible clumps remained. The tubes were incubated at 37° C. for 3 h. The samples were further diluted and 100 μl were spread in duplicates onto MRS agar plates which were incubated for 48 h at 37° C. in anaerobic chambers. The regeneration and viability of the probiotics were determined by counting the colonies on the plates after 48 h of incubation, in case of slow colony growth incubation was extended to 72 h or even 96h. For the experimental setup, see FIG. 3. Results are expressed as mean values with standard deviation of colony-forming units (CFU) per millilitre calculated from the colonies formed on the agar plate.

TABLE 10
HMO compositions tested in the present example

| HMO | C | Mix1 | Mix2 | Mix3 |
|---|---|---|---|---|
| LNFP-III | 0 | 50 | 75 | 40 |
| LNnT | 0 | 50 | 25 | 55 |
| pLNnH | 0 | 0 | 0 | 5 |

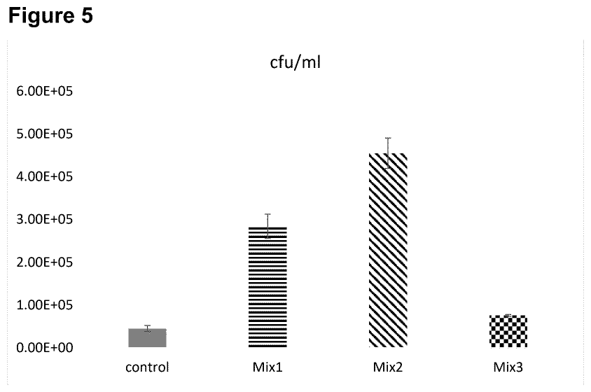

FIG. 4 shows the plates with the colonies of Lactobacillus casei formed after the 3 h the acidic treatment and 72 hours incubation. The E-3 dilution plates were used for counting and the results are shown in FIG. 5.

Lyophilized Lactobacillus casei dissolved with the HMO mixtures described herein showed a strongly significant (between P<0.01 and P<0.05) enhanced regeneration and survivability compared to control without the HMO mixtures. These data clearly show that the regeneration and viability of Lactobacillus casei after exposure to low pH conditions, such as in the stomach or in an acidic beverage, can be improved in the presence of any of the HMO mixtures. It can also be seen that the LNFP-III amount should preferably constitute at least 50% of the HMO mixture and even more preferably at least 75% of the HMO mixture.

To our knowledge it has not previously been shown that a mixture of LNFP-III and LNnT provides a benefit of improving the regeneration and survivability of probiotics in an acidic environment.

1. A genetically engineered cell capable of producing one or more fucosylated HMOs, comprising a recombinant nucleic acid sequence encoding a fucosyltransferase with α-1,3-fucosyltransferase activity, wherein the fucosyltransferase is selected from the group consisting of:
a. Camp1, with an amino acid sequence according to SEQ ID NO: 1, or a functional homologue thereof with an amino acid sequence that is at least 80% identical to SEQ ID NO: 1;
b. Parm1 with an amino acid sequence according to SEQ ID NO: 2, or a functional homologue thereof with an amino acid sequence that is at least 80% identical to SEQ ID NO: 2; and
c. Hpull1 with an amino acid sequence according to SEQ ID NO: 3, or a functional homologue thereof with an amino acid sequence that is at least 80% identical to SEQ ID NO: 3, and
d. Med1 with an amino acid sequence according to SEQ ID NO: 4, or a functional homologue thereof with an amino acid sequence that is at least 80% identical to SEQ ID NO: 4.

2. The genetically engineered cell according to claim 1, wherein the fucosyltransferase has low or no activity on a glucose moiety in the reducing end of an acceptor molecule used to produce the one or more fucosylated HMOs.

3. The genetically engineered cell according to claim 1, wherein the one or more fucosylated HMOs produced by the cell is LNFP-III.

4. The genetically engineered cell according to claim 1, wherein the genetically engineered cell comprises a recombinant nucleic acid sequence encoding a β-1,4-galactosyltransferase.

5. The genetically engineered cell according to claim 4, wherein the genetically engineered cell further comprises a recombinant nucleic acid sequence encoding a β-1,3-N-acetyl-glucosaminyltransferase.

6. The genetically engineered cell according to claim 5, wherein the β-1,3-N-acetylglucosaminyltransferase is from Neisseria meningitidis, and the β-1,4-galactosyltransferase is from Helicobacter pylori.

7. The genetically engineered cell according to claim 1, wherein the cell comprises two or more genomic copies or a plasmid-borne copy of the recombinant nucleic acid sequence.

8. The genetically engineered cell according to claim 1, wherein the engineered cell is selected from the group consisting of Escherichia Coli, Bacillus subtilis, Lactobacillus lactis, Corynebacterium glutamicum, Yarrowia lipolytica, Pichia pastoris, and Saccharomyces cerevisiae.

9. A method for producing one or more fucosylated HMOs comprising the steps of:
a. providing the genetically modified cell according to claim 1,
b. culturing the cell according to (a) in a suitable cell culture medium to produce the one or more fucosylated HMOs, and
c. optionally, purifying the one or more fucosylated HMOs.

10. The method according to claim 9, wherein the one or more fucosylated HMOs comprises LNFP-III; and wherein at least 30% of the molar content of the total HMOs produced is LNFP-III.

11. The method according to claim 9, wherein at least 90% of the molar content of the total HMOs produced by the cell is LNFP-III and LNnT.

12. The method according to claim 9, wherein the one or more fucosylated HMOs comprises LNFP-III; and wherein the LNFP-III produced is at least 90% pure.

13. (canceled)

14. A mixture of HMOs, wherein the mixture is selected from the group consisting of:
a. 30-55 molar % LNFP-III, 40-65 molar % LNnT and 2-8% molar % pLNnH, in total adding up to 100% molar content;
b. 30-99 molar % LNFP-III, 0-65 molar % LNnT, 0-20 molar % 3-FL, 0-10 molar % LNDFH-III and 0-8 molar % pLNnH, in total adding up to 100% molar content;
c. 70-99 molar % LNFP-III and 1-50 molar % LNnT in total adding up to 100% molar content;
d. 80-99 molar % LNFP-III, 0-5 molar % LNnT and 0-10 molar % LNDFH-III, in total adding up to 100% molar content; and
e. 80-99 molar % LNFP-III and 0-20 molar % 3-FL in total adding up to 100% molar content.

15. (canceled)

16. The genetically engineered cell according to claim 4, wherein the β-1,4-galactosyltransferase is from Helicobacter pylori.

17. The genetically engineered cell according to claim 5, wherein the β-1,3-N-acetylglucosaminyltransferase is from Neisseria meningitidis.

18. The method according to claim 9, wherein less than 5% of the molar content of the total HMOs produced by the method is another complex fucosylated HMO.

19. The method according to claim 9, wherein less than 3% of the molar content of the total HMOs produced by the method is LNFP-VI and/or LNDFH-III

20. The method according to claim 9, wherein at least 30% of the molar content of the total HMOs produced; and less than 5% of the molar content of the total HMOs produced by the method is another complex fucosylated HMO.

21. The method according to claim 9, wherein at least 30% of the molar content of the total HMOs produced; and less than 3% of the molar content of the total HMOs produced by the method is LNFP-VI and/or LNDFH-III.

22. The method according to claim 9, wherein at least 30% of the molar content of the total HMOs produced; less than 5% of the molar content of the total HMOs produced by the method is another complex fucosylated HMO; and less than 5% of the molar content of the total HMOs produced by said method is another complex fucosylated HMO.
